SEQUENCE LISTING (24 sequences)

SEQ ID NO: 1 - DNA, 6509 bp, Zea mays
acctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcaacctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcagcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacgacctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcagcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacgacctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcagcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacgacctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcagcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacgacctttatgatgtggctaaggctaaaacgtatgtaaaggtgaagtgatcatgcatgtcattcctcatgaattagaagaattccaatagaaagcgacactttttttcttgtatgtagtggaaactatctttccaacacaaaccatacaatccaacgaaactgagaactaaatgacaaaattgactaggagacatgtcatctctagcttatctgcttgttggcaaaatctaaaatttgaacacacatacatgagcttagacttgtataaatagggcccaaatcaatagttaatccatcgcccataacactgagatcaattaggaacataccaagtgaaacacactagcagcatcttggcaagaatggctaccaagatatttgccctccttgtgctccttgctctttcagcgagcgctacaactgcggtcattattccgcagtgctcacttgctcctactgctgctattattcagcggttcctcccacctgtttctgctatagggttcgaacacccagctgtgcaggcctacaggctacaacaggcgcttgcggcaaccatcttacaacaaccacttgcccaattgcaacaacggtcctcggctcatctaactatacagacaatcgcagcacaacaacaacaacaacagttcctggcatcactcagtcaactagcagcagccgcgaacccagctgcatacttgcaacaacagctgctggcatccaacccacttgctgtggcgaacgctattgaataccagcaacaacaacagctgcaacagttcctgccagcgctcagtcaactagtcgtggcaaaccctgctgcctatctgcaatcacaattgtttccatccaacccactagtcgtgaccaattccgctgcatacctgcagcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacggcaacagcagctacaacagatcctaccagcactcagtcaactagccgtggcgaaccctaactcctacttgcaacaacaacaacttcttccattcaaccaagtggctgtggcgaacaacgctgtataccagcaacaatatcagctgctgcaagttaacccattggttgccaccttcctacagcagcagcaacggcaactactgccattcaaccaaatgtctt

SEQ ID NO: 2 - DNA, 95 bp, Zea mays
gcaacacatatgtggctatcgttacacatgtgtaaaggtattgcatcacaccattgtcacccatgtatttggacaataccgagaggaaaacccac

SEQ ID NO: 3 - DNA, 1178 bp, zea mays
  misc_feature (21)..(39): n is a, c, g, or t
gcaacacatatgtggctatcnnnnnnnnnnnnnnnnnnnattgcatcacaccattgtcacccatgtatttggacaataccgagaggaaaacccacttatttattgtattttatcaagtttgtcttgcttacgtataaattataacccaacaaagtagtcactaaatgtcaaaaccaactagataccatgtcatctctaccttatcttactaatgttctttttgcaaaatccaaaattaatcttgcacaagcacaaggactgatatgtgtataaatatctcttagattagtagttaatacatcgcacatattattgagaccaactagcaacatagaaagcacaatagtgtaccaacaatggcagccaaaatattttgcttccttatgctccttggtctttctgcaagtgttgctaccgcaaccattttcccannnnnnnnnnnnnnnnnnnnnnnnnnnnnnnnnnnnnnnnnnnnctcaccagcggtgtcttcaatgtgtgaaaacccaattgttcaaccctacaggatccaacaggcaatcgcaacaggcatcttaccattatcacccttgttcctccaacaaccgtcagccctattacagcagttacctttggtccatttggtggcacaaaacatcagnnnnnnnnnnnnnnnnnnnnttgtgctagcaaaccttgctcgatactctcagcaacatcagnnnnnnnnnnnnnnnnnnnnnnnnncattgaactctgctgcttatttgcaacaacannnnnnnnnnnnnnnnnnnnttgctgcctacccccagnnnnnnnnnnnnnnnnnnnnnnnnncagcattgaactctgctgcttatttacagcagcaacaactacnnnnnnnnnnnnnnnnnnnngatgtgagccctgctgccttcttgacacaacaacagttgttgccgttctacctgcacgctatgcctaacgctggcaccctcttacaactgcaacaattgctgccattcaaccaacttgctttgacaaactcaacagtgttctaccaacaaccnnnnnnnnnnnnnnnnnnnnnnnnnnnnnnnnnngagttatagttcaataatgaagttttttggatgatgtttgtggcgtcccagaaataagaaagtacatttctagattcttatgtgcttctagtctccaaatgtggtcgatag

SEQ ID NO: 4 - DNA, 96 bp, zea mays
ggtattgcatcacaccattgtcacccatgtatttggacaataccgagaggaaaacccacttatttattgtattttatcaagtttctcttgcttacg

SEQ ID NO: 5 - DNA, 1177 bp, zea mays
  misc_feature (21)..(39): n is a, c, g, or t
gcaacacatatgtggctatcnnnnnnnnnnnnnnnnnnnattgcatcacaccattgtcacccatgtatttggacaataccgagaggaaaacccacttatttattgtattttatcaagtttgtcttgcttacgtataaattataacccaacaaagtagtcactaaatgtcaaaaccaactagataccatgtcatctctaccttatcttactaatgttctttttgcaaaatccaaaattaatcttgcacaagcacaaggactgatatgtgtataaatatctcttagattagtagttatacatcgcacatattattgagaccaactagcaacatagaaagcacaatagtgtaccaacaatggcagccaaaatattttgcttccttatgctccttggtctttctgcaagtgttgctaccgcaaccattttcccannnnnnnnnnnnnnnnnnnnnnnnnnnnnnnnnnnnnnnnnnnnctcaccagcggtgtcttcaatgtgtgaaaacccaattgttcaaccctacaggatccaacaggcaatcgcaacaggcatcttaccattatcacccttgttcctccaacaaccgtcagccctattacagcagttacctttggtccatttggtggcacaaaacatcagnnnnnnnnnnnnnnnnnnnnttgtgctagcaaaccttgctgcatactctcagcaacatcagnnnnnnnnnnnnnnnnnnnnnnnnncattgaactctgctgcttatttgcaacaacannnnnnnnnnnnnnnnnnnnttgctgcctacccccagnnnnnnnnnnnnnnnnnnnnnnnnncagcattgaactctgctgcttatttacagcagcaacaactacnnnnnnnnnnnnnnnnnnnngatgtgagccctgctgccttcttgacacaacaacagttgttgccgttctacctgcacgctatgcctaacgctggcaccctcttacaactgcaacaattgctgccattcaaccaacttgctttgacaaactcaacagtgttctaccaacaaccnnnnnnnnnnnnnnnnnnnnnnnnnnnnnnnnnngagttatagttcaataatgaagttttttggatgatgtttgtggcgtcccagaaataagaaagtacatttctagattcttatgtgcttctagtctccaaatgtggtcgatag

SEQ ID NO: 6 - DNA, 282 bp, Zea mays
gcaacacatatgtggctatcgttacacatgtgtaaaggtattgcatcacaccattgtcacccatgtatttggacaataccgagaggaaaacccacttatttattgtattttatcaagtttgtcttgcttacgtataaattataacccaacaaagtagtcactaaatgtcaaaaccaactagataccatgtcatctctactatcttactaatgttctttttgcaaaatccaaaattaatcttgcacaagcacaaggactgatatgtgtataaatatctcttag

SEQ ID NO: 7 - DNA, 1178 bp, Zea mays
  misc_feature (21)..(39): n is a, c, g, or t
gcaacacatatgtggctatcnnnnnnnnnnnnnnnnnnnattgcatcacaccattgtcacccatgtatttggacaataccgagaggaaaacccacttatttattgtattttatcaagtttgtcttgcttacgtataaattataacccaacaaagtagtcactaaatgtcaaaaccaactagataccatgtcatctctaccttatcttactaatgttctttttgcaaaatccaaaattaatcttgcacaagcacaaggactgatatgtgtataaatatctcttagattagtagttaatacatcgcacatattattgagaccaactagcaacatagaaagcacaatagtgtaccaacaatggcagccaaaatattttgcttccttatgctccttggtctttctgcaagtgttgctaccgcaaccattttcccannnnnnnnnnnnnnnnnnnnnnnnnnnnnnnnnnnnnnnnnnnnctcaccagcggtgtcttcaatgtgtgaaaacccaattgttcaaccctacaggatccaacaggcaatcgcaacaggcatcttaccattatcacccttgttcctccaacaaccgtcagccctattacagcagttacctttggtccatttggtggcacaaaacatcagnnnnnnnnnnnnnnnnnnnnttgtgctagcaaaccttgctgcatactctcagcaacatcagnnnnnnnnnnnnnnnnnnnnnnnnncattgaactctgctgcttatttgcaacaacannnnnnnnnnnnnnnnnnnnttgctgcctacccccagnnnnnnnnnnnnnnnnnnnnnnnnncagcattgaactctgctgcttatttacagcagcaacaactacnnnnnnnnnnnnnnnnnnnngatgtgagccctgctgccttcttgacacaacaacagttgttgccgttctacctgcacgctatgcctaacgctggcaccctcttacaactgcaacaattgctgccattcaaccaacttgctttgacaaactcaacagtgttctaccaacaaccnnnnnnnnnnnnnnnnnnnnnnnnnnnnnnnnnngagttatagttcaataatgaagttttttggatgatgtttgtggcgtcccagaaataagaaagtacatttctagattcttatgtgcttctagtctccaaatgtggtcgatag

SEQ ID NO: 8 - DNA, 392 bp, Zea mays
  misc_feature (1)..(2): n is a, c, g, or t
nnattgcatcacaccattgtcacccatgtatttggacaataccgagaggaaaacccacttatttattgtattttatcaagtttgtcttgcttacgtataaattataacccaacaaagtagtcactaaatgtcaaaaccaactagataccatgtcatctctaccttatcttactaatgttctttttgcaaaatccaaaattaatcttgcacaagcacaaggactgatatgtgtataaatatctcttagattagtagttaatacatcgcacatattattgagaccaactagcaacatagaaagcacaatagtgtaccaacaatggcagccaaaatattttgcttccttatgctccttggtctttctgcaagtgttgctaccgcaaccattttcc

SEQ ID NO: 9 - DNA, 1267 bp, Zea mays
attcatatcatgttaatgttgtcacatgtgtaaaggtgaagagatcttgcatgtcattccacgtagataaaaagaatgcatatataaaaatggcacattttcttgtaggtagtggaaagtatctttccagctaagaccatataatccgatataatccgataaaggtgataactaaatgtcgaaatcgagtagatgccatatcatctataccttatctgttgtttggaaaaaaaagacaaaatccaaaaaatttatatgagatctcacctatataaatagcgcccaaatcagtagttaatccatcacccataatattttgagcattcagaaacacaccaagcgaagcgcactagcaacgaccaaacaacaatggctaccaagatattagccctccttgcgcttcttgccctttttgtgagcgcaacaaatgcgttcattattccacaatgctcacttgctcctagtgccattattccacagttcctcccaccagttacttcaatgggcttcgaacactcagctctgcaagccaacaggctacaacaagcgcttgcggcgagcgtcttacaacaacaattgcccaattgcaacaacaatctttggcacatctaacaatacaagccatcacaacgcaacagcaacaacagttcctaccagcactgagctacctagccatggtgaaccctgccgcctacttgcaacagcagctgcttgcatccaacccacttgctctggcgaacgtagttgcaaaccagcaacaacaacagctgcaacagtttctgccagcgctcagtcaactagccatggtgaaccctgccgcctacgtacaacagcaacaactgctttcatctagcccgctcgctgtgggcaatgcacctacatacctgcaacaacaattgttgcaacagattgtaccagctctgactcaactagctgtggcaaaccctgttgcctacttgcaacagctgcttccattcaaccaactgactctgtcgaactctgctgcgtacctacaacagcgacaacagttacttaatccactggtagtggcaaacccattggtcgctgcattcctacagcagcaacaattgctgccatacaaccagttctctttgatgaaccctgtcttgtcgaggcagcaacccatcgttggaggtgccatcttttagattacatatgagatgtactcgataatggtgccttcataccggcatgtgtttcctagaaataatcaatatattgattcagatttatctcgatatatttc

SEQ ID NO: 10 - DNA, 1244 bp, Zea mays
  misc_feature: X is a, c, g, or t
attcatatcatgttaatnnnnnnnnnnnnnnnnnnnnnnnnnnnnnttgcatgtcattccacgtagataaaaagaatgcatatataaaaatggcacattttcttgtaggtagtggaaagtatctttccagctnnnnnnnnnnnnnnnnnnnnnngtgataactaaatgtcgaaatcgagtagatgccatatcatctatacnnnnnnnnnnnnnnnnnnnnnnnnnnnnnnnnnnnnnnntttatatgagatctcacctatataaatagcgcccaaatcnnnnnnnnnnnnnnnnnnnnnnatattttgagcattcagnnnnnnnnnnnnnnnnnnnnnnnnnnnnnnnnnaannnnnnnnnnnnnnnnnnnnnnnnnnnnnnnnnnnnnnnnnnnnnnnnnntnnnnnnnnnnnnnnnnnnnnnnnnnnnnnnnnnannnnnnnnnnnnnnnnnnnnntnnnnnnnnnnnnnnnnnnnnnnnnnnnnnnnnnnnnnnnnnnnnnctcagctctgcaagccaacaggctacaacaagcgcttgcggcgagcgtnnnnnnnnnnnnnnnnnnnnnnnnnnnnnnnnnnnntttggcacatctaacaatacaagccatcacaacgcaacagcaacaacagttcctaccagcactgagctacnnnnnnnnnnnnnnnnnnnnnnnnnnnnnnnnnnnnnnnnnnnnnnnnnnnnnnnnnnnnnnnnnnnnttcgaannnnnnnnnnnnnnnnnnnnnnnnnnnnnnnnnnnnnnnnnnnnnnnnnnnnnnnnnnnnncctacgtacaacagcaacaactgctttcatctagcccgctcgctgtgggcaatgcacctacatacctgcaacaacaattgttgcaacagattgtaccagctctgactcaactagctgtggcaaaccctgttgcctacttgnnnnnnnnnnnnnnnnnnnnnnnnnngactctgtcgnnnnnnnnnnnnnnnnnnnnnnnnnnnnnnnnnnnnnnnnnnnnnnnctggtagtggcaaacccattggtcgctgcattcctacagcagnnnnnnnnnnnnnnnnnnnnnnnnnnnnnnnnnnnnnnnnnnnntcttgtcgannnnnnnnnnnnnnnnnnnnnnnnnnnnnnnnnnnnnnnnnnnnnnnnnnnnnnnnntaatggtgccttcataccggnnnnnnnnnnnnnnnnnnnnnnnnnnnnnnnnnncnnnnnnnnnnnnnnnnnnnnn

SEQ ID NO: 11 - DNA, 233 bp, Zea mays
gtacctctcaccaacggtgtcttcggtatgtgaaaacccaattcttcaaccctacaggatccaacaggcaatcgcagctggcatcttacctttatcacccttgttcctccaacaatcatcagccctattacagcagttacctttggtgcatttattggcacaaaacatcagggcacaacaactacaacaacttgtgctagcaaaccttgctgcctactctcagcaacagcagt

SEQ ID NO: 12 - DNA, 76 bp, Zea mays
tagttgtaattcaataataaagttttttggatgatgtatgtggccaaccagaaataagaagttacatttccagatt

SEQ ID NO: 13 - DNA, 179 bp, Zea mays
ttgctaccattctacccacagaatgtggcaaacattgttgccttcttacaacaacaacaattgctgccatttagccaacatgctttgacgaatcctaccaccttattgcaaccgcccaccattggtggtgccatcttctagattttttatgatttatactgtaataataaagttctcat

SEQ ID NO: 14 - DNA, 79 bp, Zea mays
ctacaaaccattgcaacgcaacaacaacaacaacagtttctgccatcactgagccacctagccgtggtgaaccctgtca

SEQ ID NO: 15 - DNA, 140 bp, Zea mays
cctacgtacaacagcaacaactgctttcatctagcccgctcgctgtgggcaatgcacctacatacctgcaacaacaattgttgcaacagattgtaccagctctgactcaactagctgtggcaaaccctgttgcctacttg

SEQ ID NO: 16 - DNA, 199 bp, Zea mays
gcaacacatatgtggctatcgttacacatgtgtaaaggtattgcatcacaccattgtcacccatgtatttggacaataccgagaggaaaaaccacttatttattgtattttatcaagtttgtcttgcttacgtataaattataacccaacaaagtaatcactaaatgtcaaaaccaactagataccatgtcatctctac

SEQ ID NO: 17 - DNA, 1180 bp, Zea mays
gcaacacatatgtggctatcgttacacatgtgtaaaggtattgcatcacaccattgtcacccatgtatttggacaataccgagaggaaaaaccacttatttattgtattttatcaagtttgtcttgcttacgtataaattataacccaacaaagtaatcactaaatgtcaaaaccaactagataccatgtcatctctaccttatcttactaatattctttttgcaaaatccaaaattaatcttgcacaagcacaaggactgagatgtgtataaatatctcttaaattagtagctaatatatcacacatattattgagaccaactagcaacatagaaagcacaatagtgtaccaacaatggcagctaaaatattttgcctccttatgctccttggtctttctgcaagtgctgctacggcgaccattttcccacaatgctcacaagctcctatagcttcccttcttcccccgtacctctcaccaacggtgtcttcggtatgtgaaaacccaattcttcaaccctacaggatccaacaggcaatcgcagctggcatcttacctttatcacccttgttcctccaacaatcatcagccctattacagcagttacctttggtgcatttattggcacaaaacatcagggcacaacaactacaacaacttgtgctagcaaaccttgctgcctactctcagcaacagcagtttcttccattcaaccaactagctgcattgaactctgcttcttatttgcaacaacaacaactaccattcagccagctatctgctgcctacccccagcaatttcttccattcaaccaactgacagcattgaactctcctgcttatttacagcagcaacaactactaccattcagccagctagctggtgtgagccctgctaccttcttgacacaaccacagttgttgccgttctaccagcacgctgcgcctaacgctggcaccctcttacaactgcaacaattgctgccattcaaccaacttgctttgacaaacccagcagcattctaccaacaacccatcattggtggtgccctcttttagatttcttatgagttatagttcaataataaagttttttgtctgatgtttgtggcttcccagaaataagaaagtacatttctagattcttatgtgcttctagtctccaaatgtggttgata

SEQ ID NO: 18 - DNA, 1134 bp, Zea mays
  misc_feature: X is a, c, g, or t
gcagcacctatgtggctaatgttacacatgtgtaaaggtgaatggatcacaccattctcacccatgtatttggaaaataccatgaggaaaagatcatctattttttgtattgtatagagattgtatttcttacatataaattataacaaaataaagtggtcactaaatgtcaaagccaactagataccacatcatctctagcttatcttactaatattcttttggcaaaatctaaactcaatctacataagcacaaggtctaacatgtgtataaatacctcctagattaataggtaatccatcaaccatattattgagaccaacaagcaacatagaaagtggaatccagtagcaacaacagagcaacaatggcgaccaagatattttccctccttatgctccttgctctttctgcatgtgttgctaacgcgacaattttccctcaatgctcacaagctcctatagcttcccttcttcccccataccttccatcaatgatagcttcagtatctcaaaacccagctcttcagccctataggccagccctatctttggtgcagtcattggtacaaaccatcagggcacagcagctgcagcaactcgtgctacctgtgatcaaccaagtagctctggcaaacctttctccctactctcagcaacaacaatttcttccattcaaccaactgtctacactgaaccctgctgcttatttgcagcaacaactattaccattcagccagctagctactgcctactctcagcaacaacaacttcttccatttaaccaattggccgcactgaaccccgctgcttatttgcagcagcaaatactactaccatttagccagctagctgcagcaaaccgtgcttccttcttgacacagcaacagttgctgcctttctaccagcagtttgcggctaaccccgcaaccctcttacaactacaacaattgttgccctttgtccaacttgctttgacagaccgagcggcctcctaccaacaacacatcattggtggtgccctcttttagattgcttattagttgtaattcaataataaagttttttggatgatgtatgtggccaaccagaaataagaagttacatttccagattttaatgtgatattggtct

SEQ ID NO: 19 - DNA, 1188 bp, Zea mays
tcatatattatgtggctaagggttgcacttgtgtaaaggaaaatgggtcatgccattaacacccatatattcagaaaataccatgggaagaaaagcacctttttcttgtattttgtgcaaattgttttgccaacatataaagtgtaggagaagaaagtggccactaaatatcaaaatagactaggtaccagctcatctctagtttatcttctattagttttgttggctaaagaagaaatattgtcgatatacatggccaatcatgtgtataaatagccatcaaattactagttaatccatcacccatattattgataccaactagcaacatacatagtgaagtacactagcaacatcttagcaccaatgggagccaagatttttgccctccttgccctccttgctctttcagcaagcgctgctacctcgacttttattccacaatgctcacaacaatacctctctccggtgacagccgcgggatttcaatacccaactatacaatcctacatggtacaagaggccatccaagcaagcatcttacggtcattagcattaaccctccaacaaccatatgctctattgcaacagccatccttagtgcatctgtatctccaaagaatcgcggcacaacaactacaacaacagttgctaccaacaatcaatcaagtagttgcagcgaaccttgctgcttacctccagcaacaacagtttcttccattcaatcaactagctgggtgaaccctgctatctacttgcaggcacaacagctactaccatttaaccaacttgtcgggagcccttatgccttcttactgcaacaacagcttctgccattccatctgcaagctgtggcaaacattgttgctttcttgagacaacaacatttgttgccattttacccacaagttgtgggaaacattaatgccttcttgcaacagcaacaattgctaccattctacccacagaatgtggcaaacattgttgccttcttacaacaacaacaattgctgccatttagccaacatgctttgacgaatcctaccaccttattgcaaccgcccaccattggtggtgccatcttctagattttttatgatttatactgtaataataaagttctcatgctgatatgtgcgacctctcagtaataaagtattagagatctatattttaaattcaaatgta

SEQ ID NO: 20 - DNA, 1246 bp, Zea mays
attcatatcatgttaaggttgtcacatgtgtaaaggtgaagagatcatgcatgtcattccacataaatgaaaagaattcctatataaaaatgacatgttttgttgtaggtagtggaaattatctttccagcaaagaccatataatccgataaagctgataactaaatgtcaaaatcgagtaagtgccatatcatctatatcttatctgttgtttggaaaaagacaaaatccaaaaaaaaatatatgagatctcacatgtataaatagctcccaaatcagtagttaatacatctcccataatattttcagcattcaaaaacacaccaagcgaagcgcactagcaacgacctaacaccaatggctaccaagatattagccctccttgcgcttcttgcccttttagtgagcgcaacaaatgcgttcattattccacagtgctcacttgctcctagtgccagtattccacagttcctcccaccagttacttcaatgggcttcgaacatccagccgtgcaagcctacaggctacaactagcgcttgcggcgagcgccttacaacaaccaattgcccaattgcaacaacaatccttggcacatctaaccctacaaaccattgcaacgcaacaacaacaacaacagtttctgccatcactgagccacctagccgtggtgaaccctgtcacctacttgcaacagcagctgcttgcatccaacccacttgctctggcgaacgtagctgcataccagcaacaacaacagctgcaacagtttatcggagtgctcagtcaactagccatggtgaaccctgccgtctacctacaactactttcatctagcccgctcgcggtgggcaatgcacctacgtacctacaacaacagttgctgcaacaaattgtaccagctctgactcagctagctgtggcaaaccctgctgcctacttacaacagttgcttccattcaaccaactggctgtgtcaaactctgctgcgtacctacaacagcgacaacagttacttaatccattggcagtggctaacccattggtcgctaccttcctgcagcagcaacaacaattgctgccatacaaccagttctctttgatgaaccctgccttgcagcaacccatcgttggaggtgccatcttttagattacatatgagatgtactcgacaatggtgccctcataccgacatgtgtttcctagaaataatcaatatattgattgagatttatctcgatatattc

SEQ ID NO: 21 - DNA, 1281 bp, Zea mays
  misc_feature (1)..(64): n is a, c, g, or t
nnnnnnnnnnnnnnnnnnnnnnnnnnnnnnnnnnnnnnnnnnnnnnnnnnnnnnnnnnnnnnnnataannnnnnnnnnnnnnnnnnnnnnnnnnnnnnntgttttgttgtaggtagtggaaatnnnnnnnnnnnnnnnnnnnnnnnnnnnnnnnnnnnnnctgataactaaatgtcaaaatcgagtaagnnnnnnnnnnnnnnnnnnnnnnnnnnnnnnnnnnnnnnnnnnnnnnnnnnnnnnnnnnatatatgagatctcacannnnnnnnnnnnnnnnnnnnnnnnnnnnnnnnnacatctcccataatattttcnnnnnnnnnnnnnnnnnnnnnnnnnnnnnnnnnnnnnnnnnnnntaacacnnnnnnnnnnnnnnnnnnnnnnnnnnnnnnnnnnnnnnnnnnnnnnannnnnnnnnnnnnnnnnnnnnnnnnnnnnnnnnngnnnnnnnnnnnnnnnnnnnnnngnnnnnnnnnnnnnnnnnnnnnnnnnnnnnnnnnnnnnnnnnnnnnnnnnnnnnnntccagccnnnnnnnnnnnnnnnntagcgcttgcggcgagcgcnnnnnnnnnnnnnnnnnnnnnnnnnnnnnnnnnnnnnnnnnnnnnnnnnnnnnctacaaaccattgcaacgcaacaacaacaacaacagtttctgccatcactgagccacctagccgtggtgaaccctgtcannnnnnnnnnnnnnnnnnnnnnnnnnnnnnnnnnnnnnnnnnnnnnnnnnnnnnnctgcnnnnnnnnnnnnnnnnnnnnnnnnnnnnnnnnnatgccagtnnnnnnnnnnnnnnnnnnnnnnnnnnnnnnnntctacctacaactactttcatctagcccgctcgcggtgggcaatgcacctacgtacctacaacaacagttgctgcaacaannnnnnnnnnnnnnnnnnnnnnnnnnnnnnnnnnnnnnnnnnnnnnnnnnnnacaacagttgnnnnnnnnnnnnnnnnnnnnnntgtnnnnnnnnnnnnnnnnnnnnnnnnnnnnnnnnnnnnnnnnnnnnnnnnnnnnnnnnnnnnnnnnnnnnnnnnnnnnnnaccttcctgcagcagcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaatggtgccctcataccgannnnnnnnnnnnnnnnnnnnnnnnnnnnnnnnnnnnnnnnnnnnnnnnnnnnnnnnnn

SEQ ID NO: 22 - DNA, 1181 bp, Zea mays
  misc_feature (200)..(221): n is a, c, g, or t
gcaacacatatgtggctatcgttacacatgtgtaaaggtattgcatcacaccattgtcacccatgtatttggacaataccgagaggaaaaaccacttatttattgtattttatcaagtttgtcttgcttacgtataaattataacccaacaaagtaatcactaaatgtcaaaaccaactagataccatgtcatctctacnnnnnnnnnnnnnnnnnnnnnntgcaaaatccaaaattaatcttgcacaagcacaaggactgagatgtgtataaatatctcttaaattagtagctaatatatcacacatattattgagaccaactagcaacatagaaagcacaatagtgtaccaacaatggcagctaaaatattttgcctccttatgctccttggtctttctgcaagtgctgctacggcgaccattttcccannnnnnnnnnnnnnnnnnnnnnnnnnnnnnnnnnnnnngtacctctcaccaacggtgtcttcggtatgtgaaaacccaattcttcaaccctacaggatccaacaggcaatcgcagctggcatcttacctttatcacccttgttcctccaacaatcatcagccctattacagcagttacctttggtgcatttattggcacaaaacatcagggcacaacaactacaacaacttgtgctagcaaaccttgctgcctactctcagcaacagcagtnnnnnnnnnnnnnnnnnnnnnctgcattgaactctgcttcttatttgcaacaacaacaactaccattcagccagctatctgctgcctacccccagnnnnnnnnnnnnnnnnnnnnnnnnacagcattgaactctcctgcttatttacagcagcaacaactactaccattcagccagctagctggtgtgagccctgctaccttcttgacacaaccacagttgttgccgttctaccagcacgctgcgcctaacgctggcaccctcttacaactgcaacaattgctgccattcaaccaacttgctttgacaaacccagcagcattctaccaacaaccnnnnnnnnnnnnnnnnnnnnnnnnnnnntcttatgagttatagttcaataataaagttttttgtctgatgtttgtggcttcccagaaataagaaagtacatttctagattcttatgtgcttctagtctccaaatgtggttgatag

SEQ ID NO: 23 - DNA, 1192 bp, Zea mays
  misc_feature (201)..(221): n is a, c, g, or t
gcagcacctatgtggctaatgttacacatgtgtaaaggtgaatggatcacaccattctcacccatgtatttggaaaataccatgaggaaaagatcatctattttttgtattgtatagaattgtatttcttacatataaattataacaaaataaagtggtcactaaatgtcaaagccaactagataccacatcatctctagnnnnnnnnnnnnnnnnnnnnnggcaaaatctaaactcaatctacataagcacaaggtctaacatgtgtataaatacctcctagattaataggtaatccatcaaccatattattgagaccaacaagcaacatagaaagtggaatccagtagcaacaacagagcaacaatggcgaccaagatattttccctccttatgctccttgctctttctgcatgtgttgctaacgcgacaattttccctnnnnnnnnnnnnnnnnnnnnnnnnnnnnnnnnnnnnnnnnnnnntccatcaatgatagcttcagtatgtgaaaacccagctcttcagccctataggctccaacaagcaatcgcagcaagcaacatacctttatcacccttgttgtttcaacaatcgccagccctatctttggtgcagtcattggtacaaaccatcagggcacagcagctgcagcaactcgtgctacctgtgatcaaccaagtagctctggcaaacctttctccctactctnnnnnnnnnnnnnnnnnnnnnnnnnnnnnnnnntctacactgaaccnnnnnnnnnnnnnnnnnnnnnnnntattaccattcagccagctagctannnnnnnnnnnnnnnnnnnnnnacttcttccatttaaccaattggccgcactgaaccccgctgcttatttgcagcagcaaatactactaccatttagccagctagctgcagcaaaccgtgcttccttcttgacacagcaacagttgctgcctttctaccagcagtttgcggctaaccccgcaaccctcttacaactacaacaattgttgccctttgtccaacttgctttgacagaccgagcggcctcctaccaacaacannnnnnnnnnnnnnnnnnnnnnnnnnnnnnnnnntagttgtaattcaataataaagttttttggatgatgtatgtggccaaccagaaataagaagttacatttccagattttaatgtgatattggtct

SEQ ID NO: 24 - DNA, 1187 bp, Zea mays
  misc_feature (698)..(723): n is a, c, g, or t
tcatatattatgtggctaagggttgcacttgtgtaaaggaaaatgggtcatgccattaacacccatatattcagaaaataccatgggaagaaaagcacctttttcttgtattttgtgcaaattgtttgccaacatataaagtgtaggagaagaaagtggccactaaatatcaaaatagactaggtaccacgtcatctctagtttatcttctattagttttgttggctaaagaagaaatattgtcgatatacatggccaatcatgtgtataaatagccatcaaattactagttaatccatcacccatattattgataccaactagcaacatacatagtgaagtacactagcaacatcttagcaccaatgggagccaagatttttgccctccttgccctccttgctctttcagcaagcgctgctacctcgacttttattccacaatgctcacaacaatacctctctccggtgacagccgcgggatttcaatacccaactatacaatcctacatggtacaagaggccatccaagcaagcatcttacggtcattagcattaccctccaacaaccatatgctctattgcaacagccatccttagtgcatctgtatctccaaagaatcgcggcacaacaactacaacaacagttgctaccaacaatcaatcaagtagttgcagcgaaccttgctgcttacctcnnnnnnnnnnnnnnnnnnnnnnnnnntcaactagctggggtgaaccctgctatctacttgcaggcacaacagctactaccatttaaccaacttgtcgggagcccttatgccttcttactgcaacaacagcttctgccattccatctgcaagctgtggcaaacattgttgctttcttgagacaacaacatttgttgccattttacccacaagttgtgggaaacattaatgccttcttgcaacagcaacaattgctaccattctacccacagaatgtggcaaacattgttgccttcttacaacaacaacaattgctgccatttagccaacatgctttgacgaatcctaccaccttattgcaaccgcccaccattggtggtgccatcttctagattttttatgatttatactgtaataataaagttctcatgctgatatgtgcgacctctcagtaataaagtattagagatctatattttaaattcaaatgta

(19) United States
(12) Patent Application Publication
(10) Pub. No.: US 2008/0215251 A1
(43) Pub. Date: Sep. 4, 2008

(54) Computational method for choosing nucleotide sequences to specifically silence genes

(75) Inventor: David Selinger, Johnston, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., Johnston, IA (US)

(21) Appl. No.: 11/823,824

(22) Filed: Jun. 28, 2007

Related U.S. Application Data
(60) Provisional application No. 60/841,572, filed on Aug. 31, 2006.

(51) Int. Cl. G01N 33/48; G06F 3/048

(52) U.S. Cl. 702/20; 715/781

(57) ABSTRACT

A method for identifying subsequences in a polynucleotide sequence for specifically silencing a target gene is provided. The method is described for identifying sequences effective in silencing a target gene or a series of genes, but not others. Subsequences can be identified and scored using comparisons based on percent sequence identity with respect to a target reference sequence and siRNA algorithm analysis. The resulting subsequences may be ranked based on score, percent sequence identity. The identification of subsequences may be performed using a sliding window to identify all subsequences of a set length within the sequence. A user interface may be provided for displaying the results to a user.

### CROSS-REFERENCE TO RELATED APPLICATIONS

This application claims priority under 35 U.S.C. § 119 of a provisional application Ser. No. 60/841,572 filed Aug. 31, 2006, which application is hereby incorporated by reference in its entirety.

### FIELD OF THE INVENTION

The present invention generally relates to the field of biotechnology and molecular biology and to the use of computational tools for analyzing nucleic acid sequences. More particularly, the present invention relates to computer- and software-based tools for identifying a sequence for specifically silencing a target sequence.

### BACKGROUND OF THE INVENTION

Post-transcriptional gene silencing (PTGS) or RNA interference (RNAi) can arise as a result of one or more of several mechanisms, including, for example, through the use of double stranded RNAs (ds RNA) referred to as short interfering RNAs (siRNAs). siRNAs can be used to “silence” a gene either fully or partially. Since RNA is only found in cells as single-stranded, the presence of dsRNA essentially triggers a protection mechanism in the cell. An enzyme, Dicer, in the cell recognizes the dsRNA and cleaves it into siRNAs, typically between 19-25 base pairs in length. One of the strands of the siRNA becomes incorporated into the cell's RNA Induced Silencing Complex (RISC) and binds to the complementary mRNA. The bound mRNA is cleaved by an enzyme in the RISC, resulting in decreased expression levels of the cognate protein. Thus, RNA can result in expression of a particular gene being completely or partially suppressed.

Suitable target genes for silencing will occur to those skilled in the art as appropriate to the problem in hand. For instance, in plants, it may be desirable to silence genes conferring unwanted traits in the plant by transformation with transgene constructs containing elements of these genes. Examples of this type of application include silencing of genes involved in pollen formation so that breeders can reproducibly generate male sterile plants for the production of hybrids; silencing of genes involved in regulatory pathways controlling development or environmental responses to produce plants with novel growth habits or disease resistance, including the modulation of metabolic pathways to alter compositions of protein, oil, and starch components in the plant or parts thereof, for example, the seed.

One problem which exists in actually utilizing efficient gene silencing is identifying appropriate sequences to specifically target a gene. Currently, the identification of sequences for use in gene silencing applications is largely empirical. The silencing sequence is selected based on the shared percent identity of the sequence with the target sequence and its lack of identity with non-target sequences using a database search. This approach does not take into consideration that sequences with lower homologies may still be efficacious in silencing a non-targeted gene. The use of unpredictable sequences for silencing is not efficient or economical. For these and other reasons, there is a need for the present invention.

### BRIEF SUMMARY OF THE INVENTION

According to one aspect, a method of identifying one or more polynucleotide sequence for specifically silencing a target gene is provided. The method includes providing a target polynucleotide sequence to be silenced and processing the polynucleotide sequence into a series of polynucleotide subsequences. The method also provides for comparing each polynucleotide subsequences to the target sequence to obtain a percent identity for each subsequence, comparing said percent identity of each subsequence to a threshold percent identity value. The method further includes selecting each polynucleotide subsequence that meets or exceeds the threshold percent identity value, scoring each polynucleotide subsequence for potential silencing efficacy of the target polynucleotide to obtain a score, and reporting the subsequences that meet or exceed the threshold percent identity value and the score for each polynucleotide sequence that meets or exceeds the threshold percent identity value to thereby assist in identifying one or more polynucleotide subsequences for specifically silencing a target gene.

According to another aspect, a method for identifying one or more polynucleotide sequence for specifically silencing a target gene includes providing a target polynucleotide sequence to be silenced, determining a plurality of polynucleotide subsequences from the target polynucleotide sequence, determining a percent identity between each of one or more of the plurality of polynucleotide subsequence and a reference sequence, scoring each of the plurality of polynucleotide subsequences for potential silencing efficacy to provide a score for each of one or more of the plurality of polynucleotide subsequences, and reporting the score and the percent identity for at least one of the plurality of polynucleotide subsequences.

According to another aspect, a computer-implemented method of identifying one or more polynucleotide sequence for specifically silencing a target gene is provided. The method includes receiving a selection of a target polynucleotide sequence to be silenced from a user, determining a plurality of polynucleotide subsequences from the target polynucleotide sequence, determining a percent identity between each of one or more of the plurality of polynucleotide subsequence and a reference sequence, scoring each of the plurality of polynucleotide subsequences for potential silencing efficacy to provide a score for each of one or more of the plurality of polynucleotide subsequences, and providing an output to the user indicating the score for each of the one or more of the plurality of polynucleotide subsequences.

According to another aspect, a method of providing a user interface is provided. The method includes providing a display having (a) a first region adapted for displaying an identifier for each of a plurality of sequences and a score for each of the plurality of sequences, and (b) a second region adapted for displaying a markup sequence formed by marking up a target polynucleotide sequence with one of the plurality of sequences. The method provides for receiving a selection of one of the plurality of sequences from a user. The method further provides for updating the second region with the selection of the one of the plurality of sequences to display marking up of the target polynucleotide sequence with the selection of one of the plurality of sequences from the user.

The file of this patent contains a least one drawing executed in color. Copies of this patent with color drawings will be provided by the United States Patent and Trademark Office upon request and payment of the necessary fee.

### BRIEF DESCRIPTION OF THE DRAWINGS

FIG. 1 is a flow chart which provides an overview of the methodology according to one embodiment of the present invention.

FIG. 2 is a flow chart illustrating one embodiment of the methodology of the present invention.

FIG. 3 is a flow chart illustrating another embodiment of the methodology of the present invention.

FIG. 4 is a block diagram illustrating a system adapted for performing the methodology of the present invention.

FIG. 5 is an information flow diagram according to one embodiment of the present invention.

FIG. 6 is a screen display according to one embodiment of the present invention.

FIG. 7 is a screen display illustrating an alignment and the selection of sequences to silence and other options.

FIG. 8 is a screen display illustrating a graphical output.

FIG. 9 is a screen display showing synchronized selection of candidate sequence regions.

FIG. 10 illustrates an alignment pane from a screen display.

FIG. 11 illustrates a sequence pane from a screen display.

FIG. 12 illustrates a cartoon pane from a screen display.

FIG. 13 illustrates a summary table pane from a screen display.

FIG. 14 illustrates a selected sequence.

FIG. 15 illustrates alignment of the best target sequences with a match-up key and an Oligo score for the promoter silencing target for 22 kDa alpha zeins promoters.

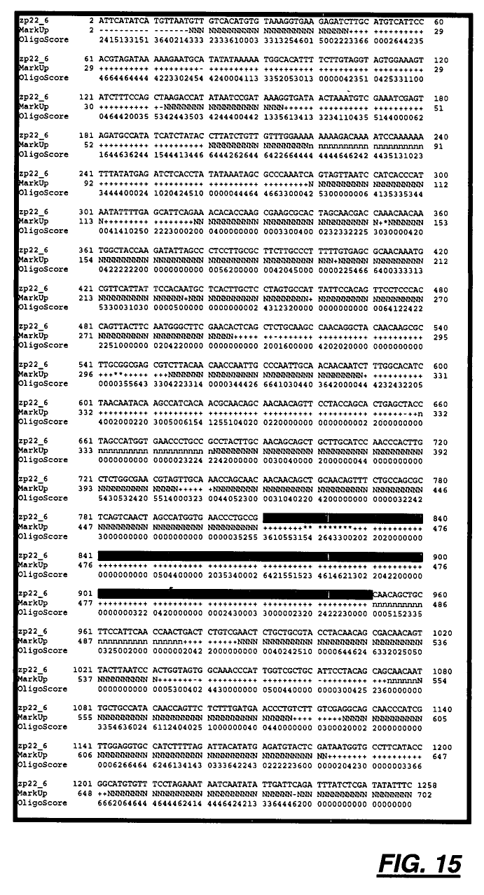

FIG. 16 a cartoon illustration of the best target sequences for the promoter silencing target for 22 kDa alpha zeins promoters.

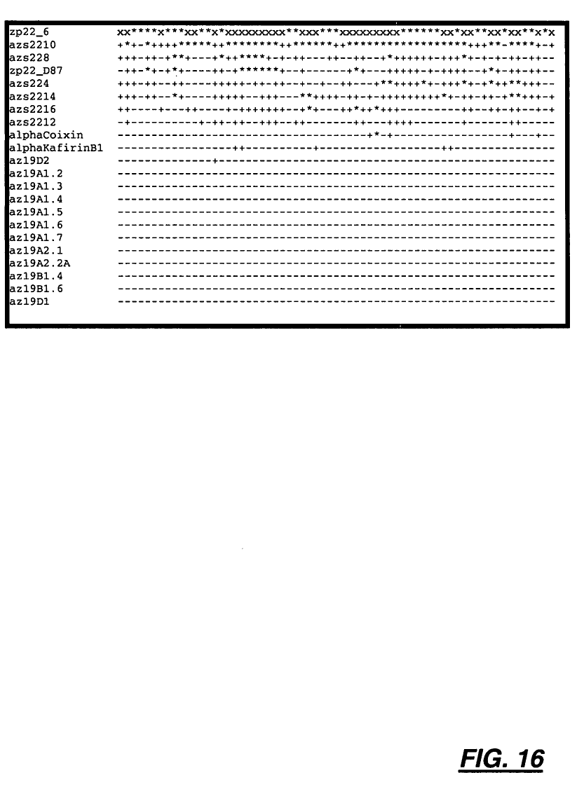

FIG. 17 is a table illustrating the best target sequences for the promoter silencing target for 22 kDa alpha zeins promoters.

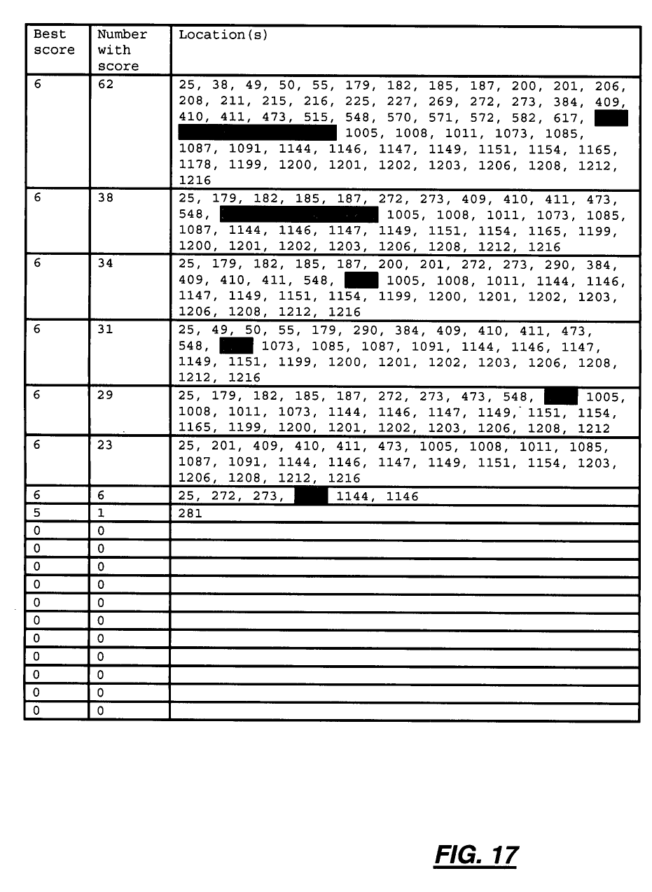

FIG. 18 illustrates zp22_{—}6 marked for matches.

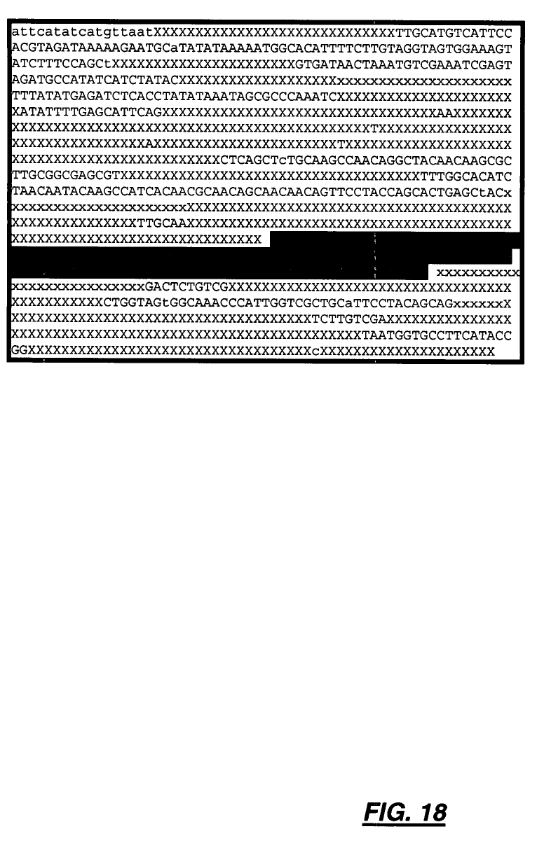

FIG. 19 illustrates recommended construct sequences.

FIG. 20 illustrates alignment of the best target sequence with a match-up key and an Oligo score where the following sequences were targeted for silencing: az19A1.2, az19A1.3, az19A1.4, az19A1.5, az19A1.6, az19A1.7, az19A2.1, az19A2.2A.

FIG. 21 provides a cartoon alignment for the best target sequences.

FIG. 22 provides a table illustrating the best target sequences.

FIG. 23 illustrates az19A 1.5 marked for matches.

FIG. 24 illustrates alignment of the best target sequence with a match-up key and an Oligo score where the following sequences were targeted for silencing: az19B1.4, az19B1.6.

FIG. 25 provides a cartoon alignment for the best target sequences.

FIG. 26 provides a table illustrating the best target sequences.

FIG. 27 illustrates az19B1.4 marked for matches.

FIG. 28 illustrates alignment of the best target sequence with a match-up key and an Oligo score where the following sequences were targeted for silencing: az19B1.4, az19D1, az19D2.

FIG. 29 provides a cartoon alignment for the best target sequences.

FIG. 30 provides a table illustrating the best target sequences.

FIG. 31 illustrates az19D1 marked for matches.

FIG. 32 illustrates alignment of the best target sequence with a match-up key and an Oligo score where the azs2216 sequence was targeted for silencing.

FIG. 33 provides a cartoon alignment for the best target sequences.

FIG. 34 illustrates azs2216 marked for matches.

### DETAILED DESCRIPTION OF THE PREFERRED EMBODIMENT

The present invention includes a method that mimics the cell's in vivo silencing process, in that a longer sequence is processed into smaller subsequences for silencing. The present invention includes methods for identifying a polynucleotide sequence specific for a nucleic acid target for use in gene silencing. One method provides for identifying subsequences within a sequence for silencing a target polynucleotide. The basic steps involved in the method involve processing a sequence into a series of overlapping, contiguous polynucleotide subsequences, comparing each of the polynucleotide subsequences to a target sequence to obtain a percent identity/similarity with a target sequence, comparing the calculated percent identity of each subsequence to a selected threshold percent identity, subjecting the subsequences to an algorithm for determining silencing potential to obtain a score, comparing the calculated score of each subsequence to a selected threshold score and reporting the subsequences based on the shared identity and siRNA score. In one aspect, subsequences that meet or exceed the threshold values with respect to identity and siRNA scores are reported. In another aspect, the present method includes generating the subsequences, in vivo, through Dicer processing of a long dsRNA precursor. This method is advantageous in that it reduces the possibility of silencing non-target genes or mRNA, thereby minimizing off-target effects on non-targeted genes or their mRNA. Thus, use of the methods and system of the present invention will increase research efficiency by facilitating the selection of polynucleotide sequences for specifically silencing a target gene, as well as saving resources that would otherwise be diverted to selecting and utilizing sequences that are ineffective for specifically silencing a target gene.

### DEFINITIONS

As used herein, the term “polynucleotide” includes double or single stranded genomic and cDNA, RNA, any synthetic and genetically manipulated polynucleotide, and both sense and anti-sense strands together or individually. This includes single- and double-stranded molecules, i.e., DNA-DNA, DNA-RNA and RNA-RNA hybrids. This also includes nucleic acids containing modified bases, for example thio-uracil, thio-guanine and fluoro-uracil.

As used herein, the terms “identical” or percent “identity,” in the context of two or more nucleic acids or polypeptide sequences, refer to two or more sequences or subsequences that are the same or have a specified percentage of nucleotides that are the same as measured using a sequence comparison algorithms or by visual inspection.

As used herein, “plant” refers to a whole plant, a plant part, a plant cell, or a group of plant cells.

The term “regeneration” as used herein, means growing a whole plant from a plant cell, a group of plant cells, a plant part or a plant piece (e.g. from a protoplast, callus, or tissue part).

As used herein, the term “sliding window” includes the examination of and reference to consecutive, overlapping subsections of a sequence, herein referred to as subsequences. The subsections can be of any length and accordingly, window size can be varied according to the user's input. For example, the window may range from about 10 nucleotides to the full length of a gene, about 12 to about 25 nucleotides, usually about 50 to about 500 nucleotides, and usually about 500 to about 2000 nucleotides. These nucleotides may be synthesized, amplified or isolated and inserted into a vector or plasmid for use in silencing. According to the present invention, the subsequence may be compared to a reference sequence, for example, a target sequence, after the two sequences are optimally aligned.

### Overview

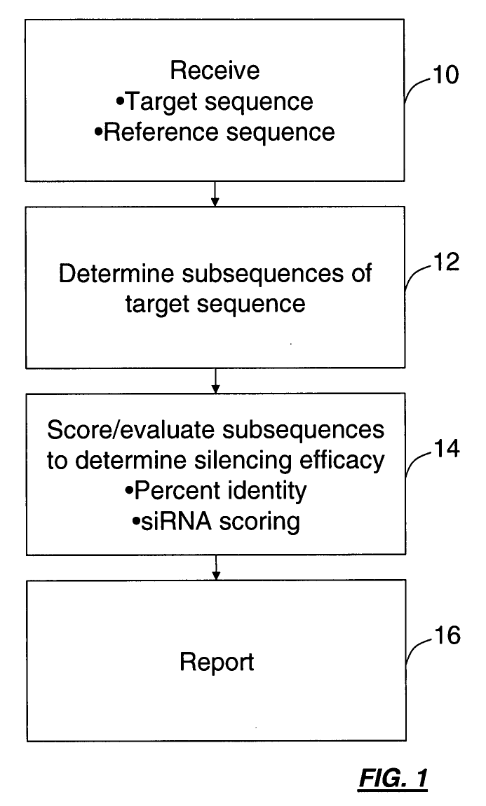

FIG. 1 provides an overview of one method. In FIG. 1, a target sequence and a reference sequence are received by a computing device in step 10. The target sequence is a sequence which is to be silenced. The reference sequence can be either a target sequence or a non-target sequence. The target sequence and/or reference sequence may be received directly from a user, come from a database, a library within a database, or elsewhere. In step 12, subsequences of the target sequence are determined. The subsequences may be determined by using a sliding window of a set length which traverses the target sequence. Each position of the sliding window results in a separate subsequence. In step 14, these subsequences are scored or otherwise evaluated to determine silencing efficacy. This can include a determination of percent identity, the use of one or more scoring algorithms such as used in siRNA scoring, or other types of scoring. Then in step 16, reporting is performed. The reporting provides indicia to a user of which subsequences are of interest such as by reporting percent identity, scores, or other information of interest.

### Providing Target Sequence and Reference Sequence

Returning to step 10, according to one aspect of the invention, the user provides at least one target sequence that he wishes to silence. The target may be endogenous with respect to the plant or a transgene, for example, a viral resistance gene, or a gene conferring resistance to nematodes. In another aspect, a user can provide multiple sequences to be targeted for silencing, for example, if one wished to identify a sequence having the ability to silence related or homologous genes or mRNA sequences or a series of dissimilar genes. The target polynucleotide sequence may be, for example, a genomic, RNA, or cDNA sequence. The provided sequence may be a full-length sequence or a partial sequence, complementary or of the same sense with respect to the target sequence that the individual wants to silence. The length of the provided sequence may be of any length, but preferably more than 19 nucleotides (nt) in length because 19 nt seems to be the shortest length of a polynucleotide that is effective for silencing a target. In one aspect, the sequence is provided by inputting the sequence into a computer program or by selecting a sequence from a database. The database may be public, for example, GenBank, PFAM or ProDom, or private. Within the database, the user may select a database, for example, for a particular library, developmental stage of an organism, a particular organism or a collection of organisms, for example, a maize genome database.

In another example, the method includes providing a non-target sequence. The non-target polynucleotide sequence may be, for example, a genomic, RNA, or cDNA sequence. The provided sequence may be a full-length sequence or a partial sequence, complementary or of the same sense as the non-target sequence that the individual does not want to silence. In one aspect, the non-target sequence is provided via user input. The user could input the sequence directly or select from a list or database. The database may be public, for example, GenBank, PFAM or ProDom, or a proprietary database. Within the database, the user may select a database, for example, a non-redundant database or a database for a library of a particular developmental stage of an organism, a particular organism or a collection of organisms. In another aspect, the user elects not to provide a non-target sequence. In another aspect, the user does not provide a non-target sequence and a default parameter for “non-target” sequences is used such that non-target sequences include all sequences other than the identified target sequence. In one aspect of the present invention, sequences may be partitioned into a subset including those to be targeted for silencing and those not targeted for silencing.

### Determining Subsequences of the Target Sequence

Returning to step 12, a method of identifying one or more polynucleotide sequences for specifically silencing a target gene includes using a sliding window analysis of a provided target sequence to generate overlapping, contiguous subsequences. The subsequences can be of any length and accordingly, the window size (length of a subsequence) can be varied according to the user's criteria or a default program parameter for length used. Generally, the window size of the selected length of the subsequence will be less than 50, 40, 30, 23, 21, 19, or 12 nucleotides. The present method analyzes all possible sequences from a target sequence for their ability to specifically silence a target gene. This is in direct contrast to other methods of siRNA design or selection that analyze the silencing potential of an individual short sequence, typically around nineteen to twenty-five nucleotides in length. By screening and identifying multiple subsequences of the target sequence, the invention increases the repertoire of available sequences that can be used for the silencing applications, thereby creating a larger pool from which to choose the better or best subsequences for silencing. In turn, this facilitates the selection of the most effective sequences for specifically silencing a target. Without wishing to be bound by this theory, the present inventors believe that the present method may be used to select subsequences more efficacious in specifically silencing a target sequence than other methods because it more closely mimics the plant cell's in vivo silencing process, using a longer sequence that is processed into smaller subsequences for silencing.

### Scoring/Evaluating Subsequences

In step 14, the sequences are scored or evaluated for silencing, shared percent identity or otherwise.

### Shared Percent Identity

A method of identifying one or more polynucleotide sequences for specifically silencing a target gene includes generating all possible subsequences of a preselected length from the provided target sequence and comparing each subsequence to the target sequence to determine the shared percent identity between the sequences. In another aspect, a method of identifying one or more polynucleotide sequences for specifically silencing a target gene includes generating all possible subsequences of a preselected length from the provided target sequence and comparing each subsequence to the non-target sequence to determine the shared percent identity between the sequences. The present invention provides for use of a computing device to align the subsequences with the reference sequence, e.g. the target sequence or the non-target sequence, and to calculate the shared sequence identity for all comparisons using algorithms designed to measure identity between two or more sequences. The shared sequence identity may be expressed as a percentage to quantitatively express the percent identity of the aligned sequences. The subsequences may be compared to the reference sequence either simultaneously or individually.

Alignment comparisons may be performed using algorithms that use a global comparison method and/or a local comparison method. In a global comparison method, the entire pair of sequences are aligned and scored in a single operation (Needlman and Wunsch), and in a local comparison method, only highly similar segments of the two sequences are aligned and scored and a composite score is computed by combining the individual segment scores, e.g., the FASTA method (Pearson and Lipman), the BLAST method (Altschul) and the BLAZE method (Brutlag). Default program parameters of these sequence algorithm programs may be used or alternatively parameters can be designated by the user. Based on the program parameters, the program's comparison algorithm calculates the percent sequence identities for the subsequences relative to the reference sequence.

The threshold shared percent identity value may be predetermined by the user, although this is not required as a default parameter can alternatively be used. Subsequences that have a percent identity value meeting the designated threshold shared percent identity value, for example, 90% identity, may be identified. The method of the present invention enables the identification of subsequences for specifically silencing a target polynucleotide without the need for performing unnecessary analysis on subsequences that do not meet the threshold requirements for shared identity with the target sequence and/or on subsequences that exceed the threshold requirements for shared identity with the non-target sequence.

In one aspect, if multiple subsequences meet the designated threshold shared percent identity value with respect to the target and/or non-target sequences, then other criteria may be used to choose among the subsequences. Therefore, in another aspect of the invention, the subsequence with a shared percent identity value that meets the designated threshold shared percent identity value may be identified for further analysis in silencing a target. For example, the subsequence can be specified to have at least 80% shared identity with the target sequence and/or have less than 60% identity to the non-target sequences. The user may preselect the threshold shared percent identity value prior or subsequent to the comparison step.

In addition, the user may want to vary the threshold shared percent identity value taking into consideration the type of sequence targeted. It may be preferable that there is complete sequence identity in the subsequence, although total complementarity or similarity of sequence is not essential. For example, biological evidence suggests that a certain level of mismatches can be tolerated by RISC relative to the mRNA targets. Therefore, a user may not require that the subsequences have high threshold shared percent identity value in all scenarios.

Further analysis of the subsequences that meet the threshold shared percent identity criteria may be undertaken to indicate which of the subsequences would be the better or best choice to use in silencing applications. A subsequence that has high threshold shared percent identity value with respect to a target sequence does not indicate that the subsequence will necessarily be effective in silencing the target sequence because other attributes of the subsequence should be considered to determine those subsequences likely to have the proper strand incorporated into the RISC complex. Thus, in one embodiment, an siRNA algorithm may be used to further evaluate the subsequences for predicted efficacy in silencing a target. The method of the present invention enables identification of subsequences for specifically silencing a target gene without the need for unnecessary siRNA algorithm analysis of subsequences not meeting or exceeding the shared percent identity threshold.

In another variation, the siRNA algorithm is used to evaluate the subsequence's predicted efficacy in silencing a target prior to determining the shared percent identity between the subsequences and the reference sequence. The methodology enables identification of subsequences for specifically silencing a target gene without the need for unnecessary shared percent identity analysis of subsequences not meeting or exceeding the siRNA efficacy threshold.

### Evaluating Sequence for Silencing Capability

Thus, in one aspect, the method of the present invention determines if the subsequences would likely be incorporated into the RISC complex. Potential silencing efficacy may be determined using an siRNA algorithm that takes into consideration a physical characteristic of the subsequence. Surprisingly, siRNA algorithms have not been used to determine the “best” sequence for silencing a target from a long sequence, typically, they are applied to an individual sequence of less than thirty nucleotides in length. In the method of identifying one or more polynucleotide sequences for specifically silencing a target gene, the sequences may be analyzed using an siRNA algorithm of, for example, a free energy differential (5′ ΔΔG), Ui-Tei et al. (Guidelines for the Selection of Highly Effective siRNA Sequences for Mammalian and Chick RNA Interference. Nucleic Acids Research. 2004. 32(3): 936-948); Hsieh et al. (A Library of siRNA Duplexes Targeting the Phosphoinositide 3-Kinase Pathway: Determinants of Gene Silencing for Use in Cell-based Screens. Nucleic Acids Research. 2004. 32(3):893-901.), Reynolds et al. (Rational siRNA Design for RNA Interference. Nat Biotechnol. 2004. 22(3):326-30), Takasaki et al. (An Effective Method for Selecting siRNA Target Sequences in Mammalian Cells. Cell Cycle. 2004. 3(6):790-95.), Amarzguioui et al., (An Algorithm for Selection of functional siRNA Sequences. Biochem Biophys Res Commun. 2004. 316(4):1050-8). Any algorithm or program may be used with the present method so long as the program is capable of evaluating whether the subsequence would likely or unlikely to be effective in silencing a particular target sequence and providing a score for a parameter that effects potential silencing efficacy of the subsequence. In one aspect of the present invention, each subsequence of the provided target sequence is subjected to an siRNA algorithm to determine its efficacy for silencing a target. Default program parameters of these sequence algorithm programs may be used or alternatively parameters can be designated by the user. Based on the program parameters, the program's algorithm scores a physical characteristic of the subsequences. In one aspect, the algorithm may determine at least one or more physical characteristics of the subsequence, including for example, its melting temperature (Tm), the nucleotide content of the 3′ overhangs, the length of the subsequence, the nucleotide distribution over the length of the subsequence, nucleotide end-composition of the target site and presence and location of mismatches with respect to a reference sequence. The value for these characteristics may be reported as a score for each subsequence. After calculating the score of the characteristic, the value of the score is analyzed to determine its value compared to a preselected threshold value. In one aspect, the value of the subsequence is greater than or equal to a preselected threshold value. In one aspect, the value of the subsequence is less than or equal to a preselected threshold value. If it is determined that all subsequences scored below the threshold, then the subsequences may be identified as being ineffective for silencing applications. If, however, there is one or more subsequences that scores above the threshold and have similar scores, then the subsequences may be further analyzed to identify its silencing efficacy. For example, selection among these subsequences may be made on the basis of other criteria, such as selecting the 3′ end of the gene that has been found to be typically more effective in silencing, determining base composition at the 5′ end of the RNA molecule, examining helix stability, determining base composition numbers at the 3′ end, in particular the frequency of A and T's in the last 7 nt at the 3′ end of the sequence, or the free energy of the molecule.

In another embodiment, the siRNA algorithm is used to evaluate the subsequence's predicted efficacy in silencing a target gene prior to determining the shared percent identity between the subsequences and the reference sequence. The method of the present invention enables identification of subsequences for specifically silencing a target gene without the need for unnecessary percent identity analysis of subsequences not meeting or exceeding the siRNA efficacy threshold.

### Reporting and Use of Results

Returning to step 16 of FIG. 1, reporting is provided. The reporting may provide various views of the matrix of what part matches from the set of A and set of B. The reporting may provide output identifying those sequences that meet the search criteria, those sequences determined to possess the preselected amount of percent identity and physical characteristics to be effective in silencing. Thus, the output can range from zero or no subsequences to multiple subsequences. The reporting may rank the subsequences or provide additional information about the subsequences as well as recommendations. For example, the reporting may indicate if a subsequence is available within an institution or commercially available. The reporting may provide additional recommendations such as increasing the length of the sequence, or other information. The present invention contemplates that multiple scoring techniques may be used and the reporting may show the results for each separate technique and/or an overall score.

After reporting, the subsequences may be used in various ways. The user may use the identified subsequences to focus on a region in the target sequence where the subsequence is localized. In another embodiment, the user may desire to use a longer sequence than the subsequence initially identified since longer sequences have been shown to be more efficacious in gene silencing in plants. As such, the user may decide to repeat the process using a longer target sequence. In another aspect, the user may decide to repeat the process using a longer subsequence, or window, than previously used. If desired, the user can input a sequence that is longer than the subsequence identified by the program. This may be undertaken to “verify” that any additional nucleotides added on to the ends of the polynucleotide subsequence would not affect the ability of the sequence to silence the target gene or inadvertently target another molecules. The subsequence may include additions at the 5′ and/or 3′ ends of the subsequence. The sequence of the nucleotides may include the nucleotides from the surrounding sequence in the target sequence or may be otherwise chosen by the user. Thus, the user may focus on the region where the subsequence is localized within the native target sequence, gene, or surrounding sequence and incorporate the surrounding nucleotides at the 5′ or 3′ end or alternately add nucleotides to the 5′ and 3′ ends of the subsequence that differ from the target sequence, gene, or surrounding sequence. In one embodiment, nucleotides are added to the subsequence such that when a RNA molecule is generated it contains inverted repeats. These inverted repeats may be used to generate a hairpin structure.

In another aspect, the present method includes generating a subsequence meeting the percent identity and siRNA potential thresholds of the method of the present invention, in vivo, through Dicer processing of a long dsRNA. The efficacy of the sequences in silencing can be confirmed using a functional assay. These sequences can then be obtained by isolation from a cell, amplified using PCR or synthesized. Such methods are routine to one skilled in the art. Once obtained the nucleic acid can be cloned into a vector using routine cloning methods in molecular biology. Any vector that is replicable and viable in the host may be employed for use with the present invention. Vectors which may be used include but are not limited to viral particles, baculovirus, phage, plasmids, phagemids, cosmids, phosmids, bacterial artificial chromosomes, viral nucleic acid, for example, vaccinia, adenovirus, foul pox virus, pseudorabies and derivatives of SV40, P1-based artificial chromosomes, yeast plasmids, yeast artificial chromosomes, and any other vectors specific for specific hosts of interest, such as bacillus, aspergillus, yeast. For example, the sequence, may clone into an expression vector downstream of a regulatory control element, for example, a promoter or enhancer, so that the double stranded RNA molecule is produced. Vectors may be obtained from commercial sources along with corresponding host cells for use in the invention. Selection of the appropriate vector and promoter is well within the level of ordinary skill in the art. In one embodiment, at least one subsequence identified by the methods discussed above may be used to generate a sense RNA molecule, an antisense RNA molecule, or a ds RNA molecule, including a dsRNA hairpin molecule, for use in silencing a target sequence. In one aspect, a molecule containing the subsequence is generated and transformed into plants. Any appropriate method of plant transformation may be used to generate plant cells containing a subsequence within the genome in accordance with the present invention. Several screening methods have been used to select from a transgenic plant population those plants in which expression of a targeted gene is suppressed. These screening methods include: 1) Visual screening of a suitable trait (e.g., flower color); 2) Quantitation of the final product of a biosynthetic pathway that includes the protein product of the targeted gene as a pathway enzyme; 3) Quantitation of the protein product of the target gene; 4) Quantitation of the mRNA product of the target gene, using Northern analysis, RNase protection assay, RT-PCR, or other suitable technique; 5) Quantitation of the transgene mRNA in vegetative tissue using Northern analysis or other suitable technique. Following transformation, plants may be regenerated from transformed plant cells and tissue.

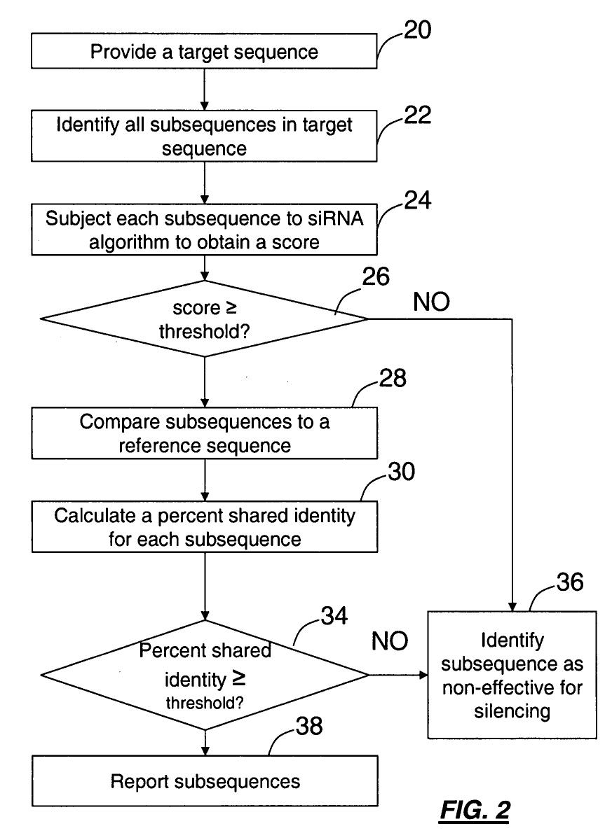

FIG. 2 illustrates one example of the methodology. In step 20, a target sequence is provided. As previously explained, the target sequence may be input by a user, selected by a user, or may be a default target sequence defined by a default variable or hard-coded into a software implemented algorithm. In step 22, all subsequences in the target sequence are identified. Although shown in FIG. 2 as a single step, the present invention may also be implemented such that one subsequence is identified at a time, scored, or otherwise evaluated. In step 24, the sequence is subjected to an siRNA algorithm to obtain a score. In step 26, a determination is made as to whether or not the score is greater than or equal to a threshold. If not, then the subsequence may be identified as being non-effective for silencing in step 36. If in step 26, the score is greater than the threshold, then in step 28, the subsequences are compared to a reference sequence. In step 30, a percent shared identity for each subsequence is determined. In step 34, a determination is made as to whether the percent shared identity is greater than or equal to a predetermined threshold. If not, then the subsequence is identified as being non-effective for silencing. If it is, then in step 38, a reporting step take place to report on the subsequences, including indicating which subsequences are effective for silencing, which are not, the scores, the percent identities, and any other observations regarding the subsequences.

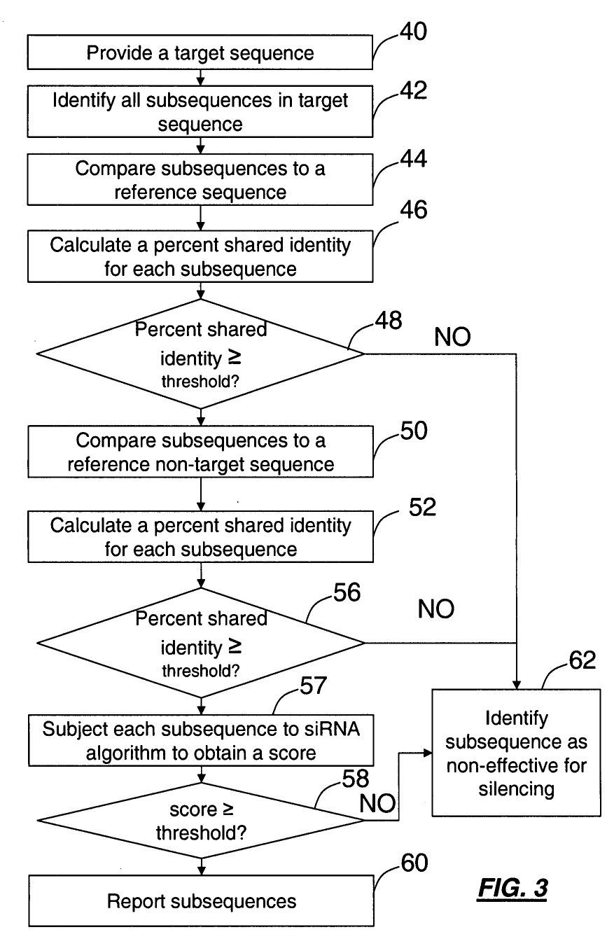

FIG. 3 illustrates another example of the methodology. In step 40, a target sequence is provided. As previously explained, the target sequence may be input by a user, selected by a user, or may be a default target sequence defined by a default variable or hard-coded into a software implemented algorithm. In step 42, all subsequences in the target sequence are identified. Although shown in FIG. 3 as a single step, the present invention may also be implemented such that one subsequence is identified at a time, scored, or otherwise evaluated. In step 44, the subsequences are compared to a reference sequence. In step 46, a percent shared identity is calculated for each subsequence. In step 48, a percent shared identity for each subsequence is compared to a threshold. If it is not greater than a predetermined threshold, then in step 62, the subsequences are identified as non-effective for silencing. If the percent shared identify is greater than or equal to the threshold then in step 50, the subsequence is compared to a reference non-target sequence. Then in step 52, a percent shared identity is calculated for each subsequence. In step 56 the percent shared identity is compared with another threshold. This may be the same threshold level as before or may be a different threshold value. If the percent shared identity is not greater than or equal to the threshold then in step 62 the subsequence is identified as being non-effective for silencing. If in step 56, the percent shared identity is greater than or equal to the threshold then in step 57, the subsequence is subjected to an siRNA algorithm to obtain a score. In step 58 the score is compared to a threshold. If the score is not greater than equal to the threshold then in step 62, the subsequence is identified as being non-effective for silencing. If it is, then in step 60 results are reported.

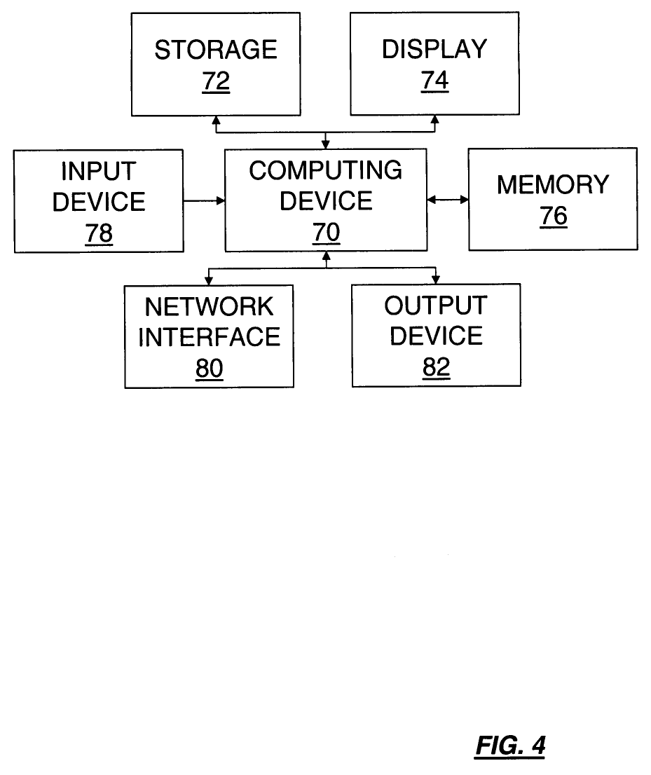

FIG. 4 illustrates one example of a computing system which is used in one embodiment of the present invention. A computing device 70 is shown which may be a personal computer or other type of computer. The computing device 70 is adapted to execute instructions to perform the determination and evaluation of sequences according to various embodiments of the present invention. The instructions may be provided in any number of computer languages, and any number of hardware or software platforms. For example, PERL may be used. Another example is that Microsoft C# may be used. The computing device is electrically connected to a storage device 72, a display 74, a memory 76, an input device 78, a network interface 80, and an output device 82. Of course, not all such components are necessary.

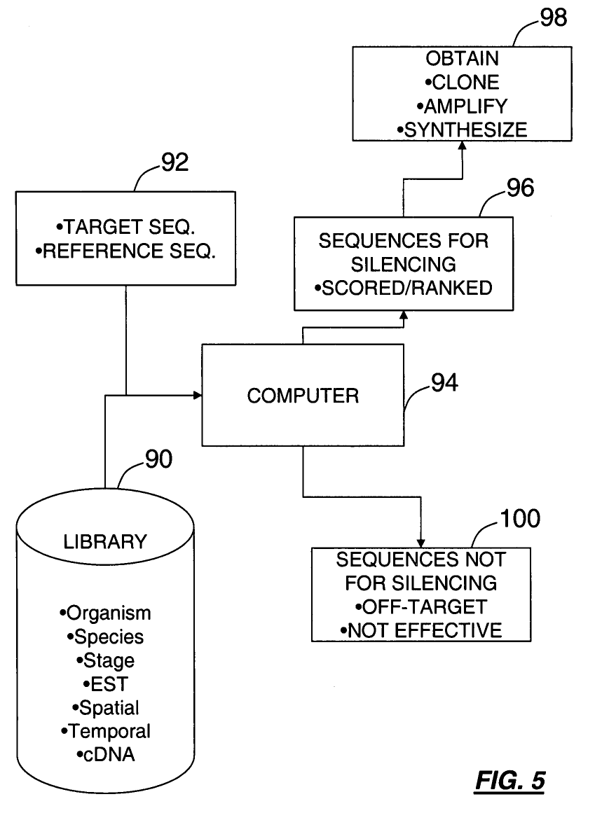

FIG. 5 illustrates information flow associated with one embodiment of the present invention. In FIG. 5, a library 90 is provided. The library contains sequences associated with organism, species, stages of an organism, expressed sequence tags (ESTs), spatial, temporal, or cDNA information. The library 90 is accessible by a computer 94. The computer 94 also receives input 92 which may include a target sequence and/or a separate reference sequence. The computer 94 performs processing and provides subsequences for silencing output 96 indicative of sequences for silencing. This may include various types of reporting including scoring, ranking, or other information. The computer 94 also provides subsequences not for silencing output 100. This may include off-target sequences, not effective sequences, or sequences which have otherwise been determined to not be effective. The present invention contemplates that once sequences for silencing are obtained in step 96, a user may use these sequences in any number of ways. A user may increase the length of these sequences and re-evaluate them, the user may obtain the sequences through cloning, amplifying, synthesizing, purchasing, or otherwise.

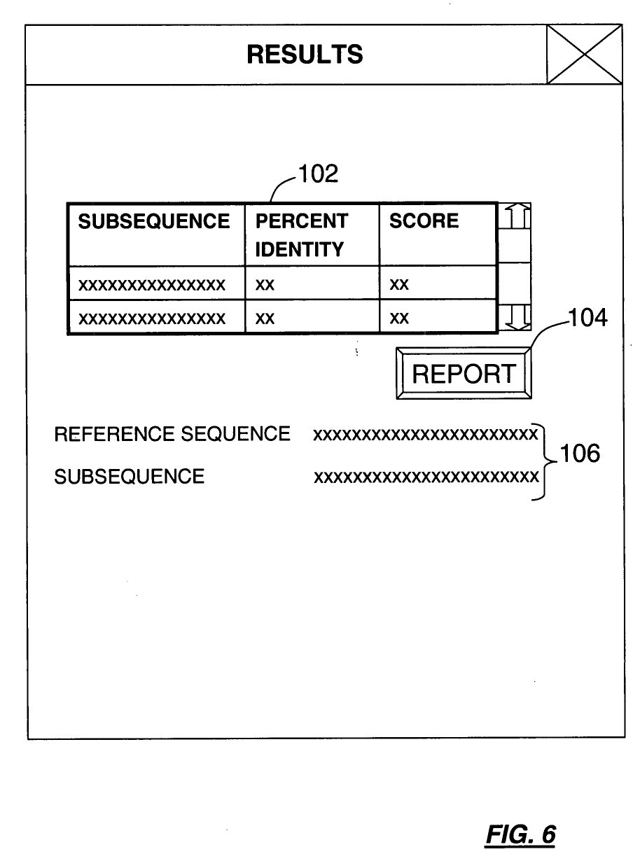

FIG. 6 provides a screen display. The screen display shown includes a table 102 which provides a listing of subsequences, percent identity for each of the subsequences and a score. The user interface, also preferably shows a comparison 106 between the reference sequence and one or more subsequences. Such a user interface allows a user to quickly see the results. The present invention also contemplates that a user may want more in-depth reporting. In such a case, the user may request such a report, such as by selecting the report button 104. The present invention contemplates that the score shown may be an overall score based on multiple scoring methodologies. So, for example, the user may select a report to see the constituent portions of each score. In addition, when the user selects a report, the report may provide additional insight to the user, such as suggesting lengthening the sequence, reporting on the commercial availability of the subsequence, or other pertinent or desirable information.

Software Implementation with User Interface

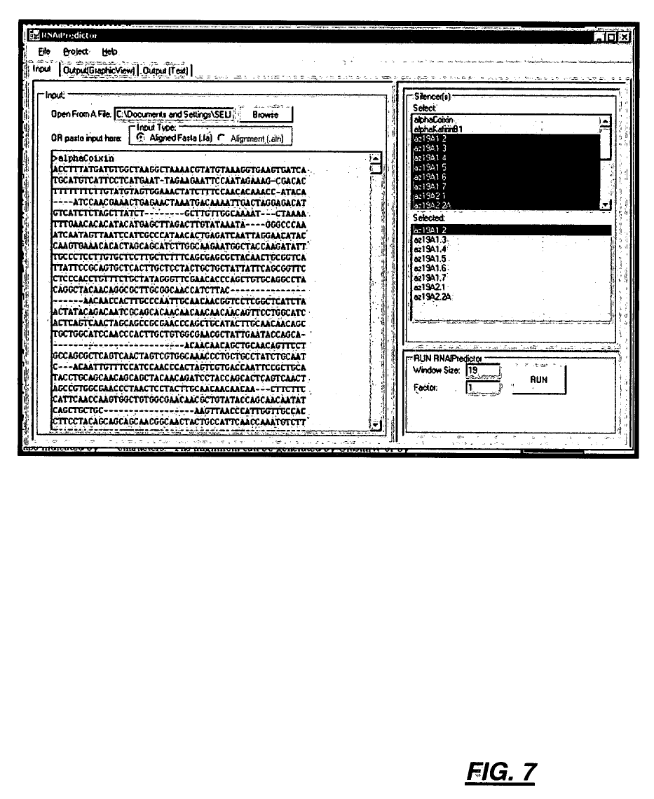

FIG. 7 through FIG. 13 illustrate a software program having a user interface which may be used. The program identifies the sequence regions that are likely to specifically silence one or more members of a gene family, while not suppressing the expression of other members. The basic idea is that silencing is based on identical short RNA segments, and this program mimics what we know of how silencing works in vivo. What we know about post-translation silencing, or RNA interference (RNAi) is that typically segments of 21-25 nucleotides in length are generated from a much longer dsRNA precursor. This precursor is directly formed from the hairpin constructs and indirectly from sense or antisense transcripts via an RNA dependent RNA polymerase. Evidence in the literature suggests that for a long precursor, the chopping process in vivo, via Dicer, begins at random locations on the dsRNA molecule, but that Dicer is processive and will then clip 21-25 nt segments after the first cut. These dsRNA segments are then unwound by the RISC complex and one of the two strands is incorporated into the RISC and trimmed to 21 nt. Apparently only 19 nt of the 21 nucleotide strand is capable of pairing with the target. The unwinding and strand choice steps depend on the base composition of the dsRNA strands in an incompletely known fashion. Some rules have been determined that discriminate between dsRNA segments where the anti-sense strand is likely to be chosen and those where the anti-sense strand is unlikely to be chosen. If the proper, anti-sense, strand is chosen, then the RISC complex is capable of cleaving mRNAs that match it. mRNA with a perfect match are targeted and there may be targeting of imperfect matches. Studies in mammals with short interfering RNAs, siRNA, indicate that as few as 12 matching nucleotides in at the 3′ end are enough to target. How well the siRNA results apply to RNAi generated from long precursors (hairpins, antisense or sense co-suppression) is not known.

As shown in FIG. 7, the program takes as input an alignment of nucleotide sequences in fasta format with gaps indicated by “−” characters or in .aln format from ClustalW. The alignment can be generated by ClustalW or by AlignX in VNTi, or by any other multiple sequence alignment tool. The alignment contains sequences from genes that are desired to be silenced and those that are not desired to be silenced.

Under the “File” menu item on the top bar of the screen display of FIG. 7 are a list of standard set of actions (not shown), such as “New”, “Import”, “Print”, and other common actions associated with Windows-based software applications. Under the “Project” menu item are actions that allow the user to open and save the project, including the current selection. Saved projects may be in an XML format and where saved in such a fashion can be moved around like any text file. When opened, the selection made before saving is displayed. Under “Help”, information about the program, its use, and examples of alignments and a project file may be made available. FIG. 7 illustrates that an input file in Aligned Fasta format has been opened.

Once the alignment has been loaded by pasting or uploading a file, then the sequence ids will show up in list box labeled “Select” as shown in FIG. 7. Selecting one or more of these sequence ids will cause them to appear in the “Selected” list box. Clicking and dragging the mouse or holding the ctrl key or shift key while clicking the mouse allows the selection of multiple ids. The “window size” and “factor” can also be adjusted. Window size controls the length of the segment used by the program to look for identical matches. Useful values may include those in the range of 19-25, or those which are smaller, including between 12-25, the value selected depending upon how stringent a user desires to be. Of course this value can vary as previously explained. The “Factor” controls how many matches starting in a window will be counted before a minimal match is shown. The default is one match starting in the window. For a window size of 19, a factor of 1 means that 1 identical match of 19 bases that starts at any of the 19 bases in a given window will result in that window being counted as a match.

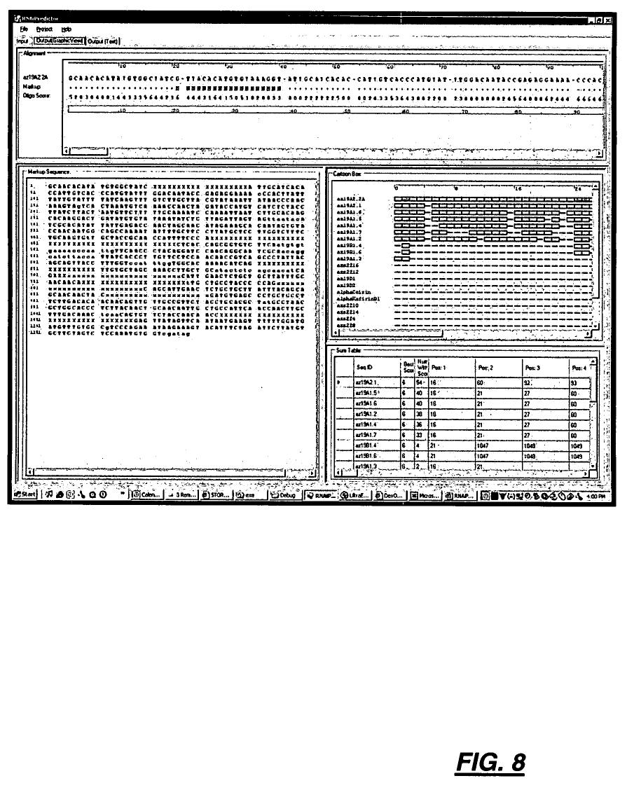

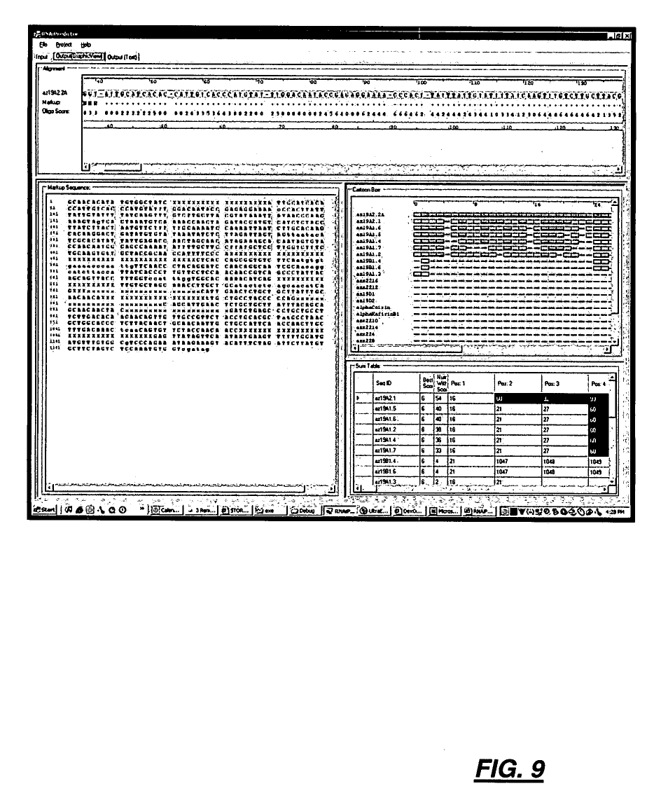

Once the “RUN” button is selected in FIG. 7, the program generates two kinds of output, graphical and text. Most of the functionality of the program is accessible from the graphical output. FIG. 8 and FIG. 9 provide screen displays of the graphical output which is divided into four panes. The panes can be resized by moving the dividing frames. The top pane shows the sequence of the “target”, which is the sequence that best matches the set of sequences to be silenced. The five lines in the pane show two different rulers (top is by character and matches the cartoon and bottom is by base and matches the sequence frame at bottom left), the sequence, whether the N-mer sequence starting at that position matches the other sequences to be silenced (+), sequences that should not be silenced (N or n) or no other sequences (−) and the silencing score for the N-mer starting at that position. The silencing score is a 0 to 6 score that is computed by a set of rules that attempt to predict how well the proper (antisense) strand will end up in the RISC complex. The bottom left pane shows the sequence after masking regions that match the non-silenced sequences and coloring the sequence that does not match the other genes to be silenced in blue. The middle right pane shows a cartoon alignment of the sequences with matching regions shown as boxes and non-matching regions shown as lines. Boxes are colored red if all N-mers in the window match the target sequence and white if at least one N-mer in the window matches. On the target sequence, blue boxes indicate matches that include sequences in the set that should not be silenced. Finally, the table in the lower right pane shows the best scoring matches in each sequence and their locations.

Selection of candidate sequence regions can be done in all four panes, and the panes are synchronized so that selection in one highlights the corresponding region in the others. Such a feature is very useful to a researcher because the different views present information in a different manner and thus it is helpful and convenient to be able to see all views at once. In the top pane and the middle right cartoon pane, selection with the mouse draws a rectangle and in the top pane selects anything the is partially covered by the rectangle. In the cartoon pane, the boxes that are completely within the rectangle are selected. However, the selection is by columns, so that selecting one box highlights the whole column. Selected regions of the cartoon are shown in red outline while in the text, the selection is shown as a gold background and in the table as a blue background. In the markup sequence pane at the lower right, selection is made by clicking and dragging the mouse and the sequence that wraps between the start and end point is selected.

Selection can also be accomplished by clicking cells in the Summary Table. Use of ctrl-click on dragging the mouse will select multiple cells. Unlike the other selection methods, this method can select discontinuous segments of sequence. Also the highlighting in the cartoon is gold rather than red. If you copy the sequence that is selected via a right mouse click (discussed in the following section), the sequence is continuous between the first and last segments. Of course, other methods of selection may be used such as may be common or customary with a user interface and other colors for the user interface may be used.

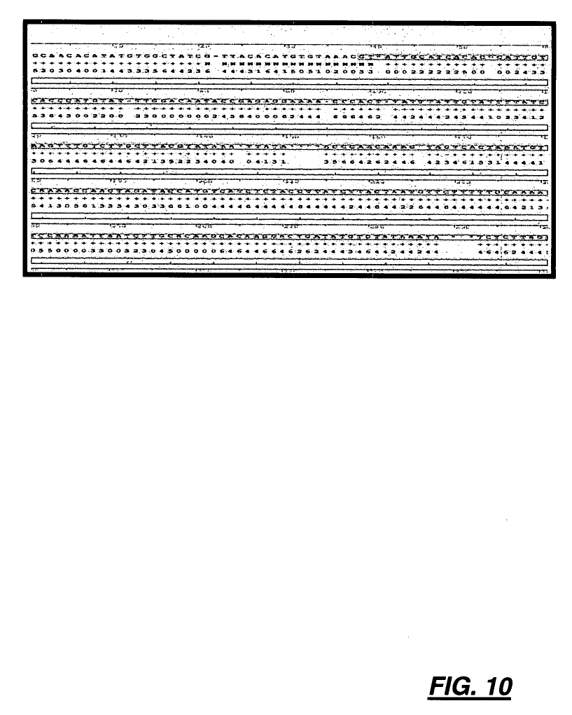

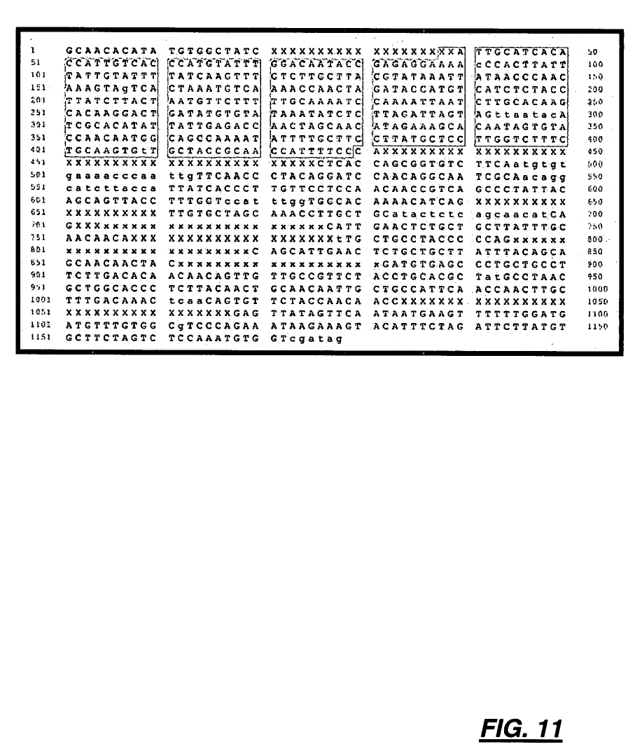

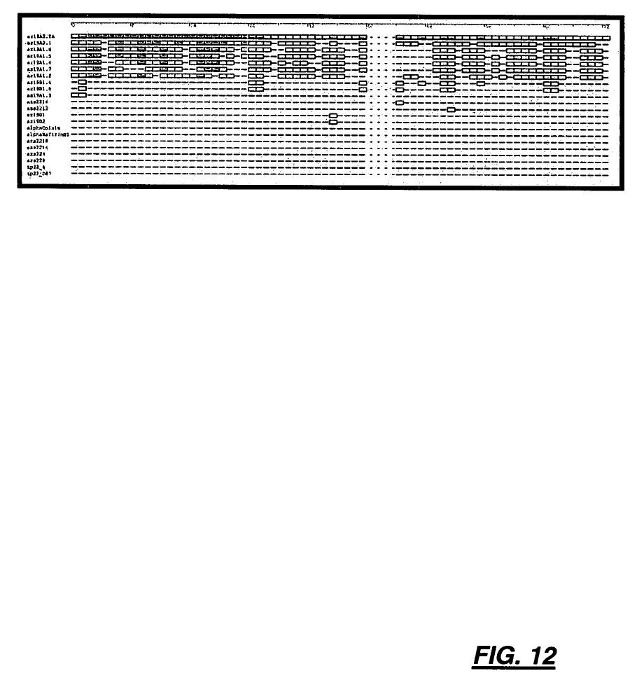

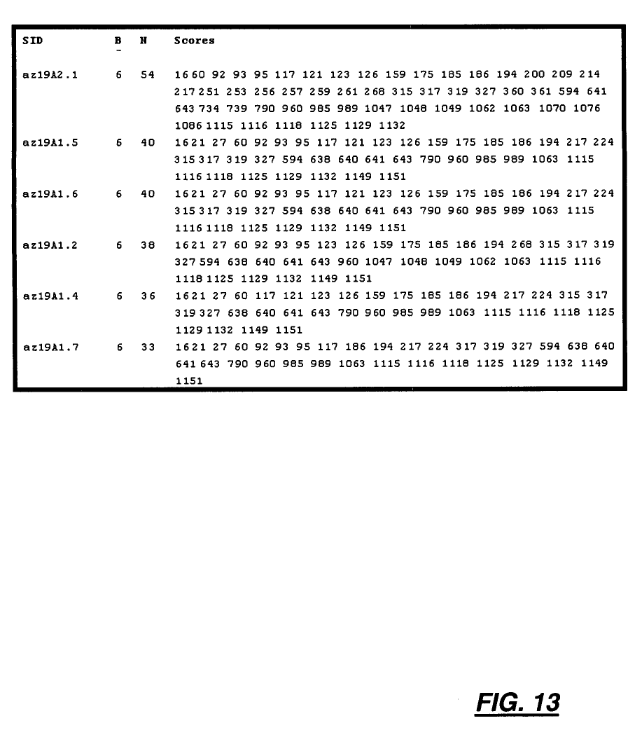

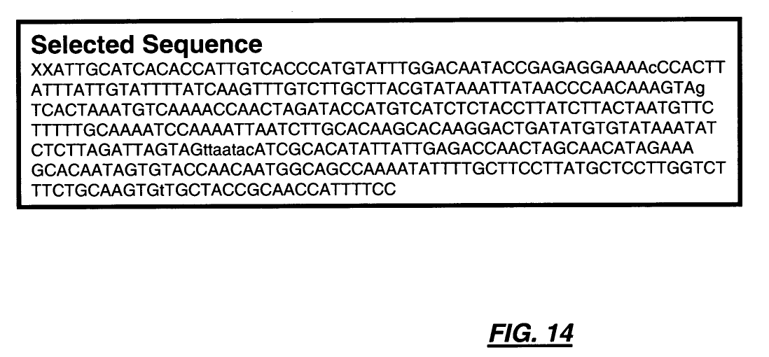

Right clicking any of the panes brings up a dialog box with the name of the pane and two options, “copy image” or “copy selected seq”. FIG. 10 through FIG. 13 provide examples of what is copied in each pane. Note that the selected region is highlighted in each of the copied images. FIG. 10 illustrates an alignment pane. FIG. 11 illustrates a cartoon pane. FIG. 12 illustrates a summary table pane. FIG. 13 illustrates a selected sequence pane. FIG. 14 illustrates a selected sequence.

### Recommended Construct Sequences

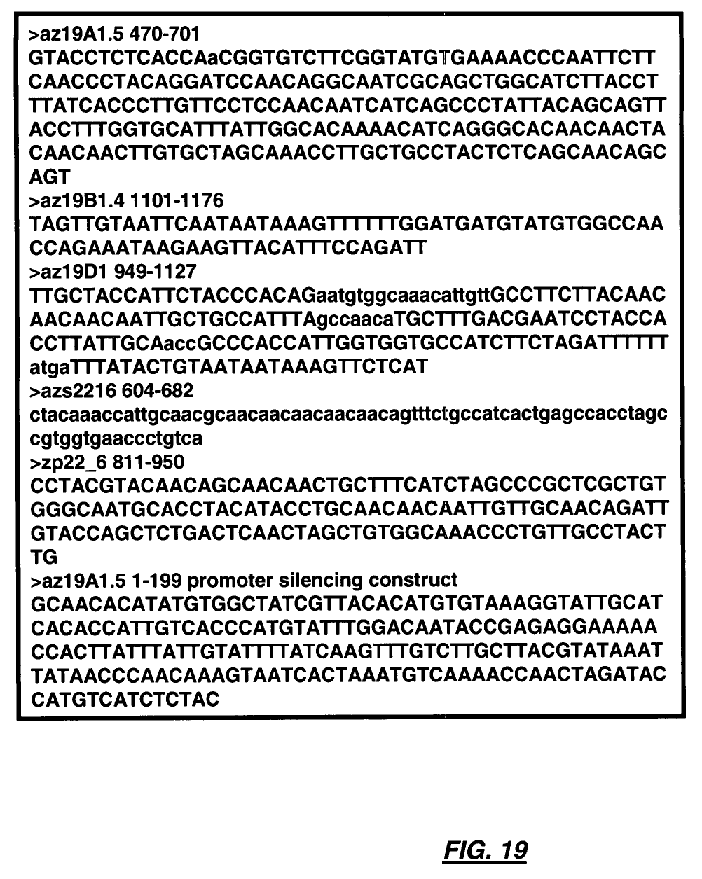

One example of an application provides for Zein Silencing Construct Planning. Based on the data in the following section these are the recommended sequences to use for each class. They should be specific to each class, should have a good chance of silencing all the members of a class and have minimal overlaps between target sequences, which should reduce or eliminate the possibility of higher order structures occurring when multiple sequences are combined into a single construct. The coordinates listed are relative to the sequences used in the overall alignment which have about 300 bases of upstream sequence. FIG. 19 illustrates the recommended sequences for each class.

19 kDa-A Class

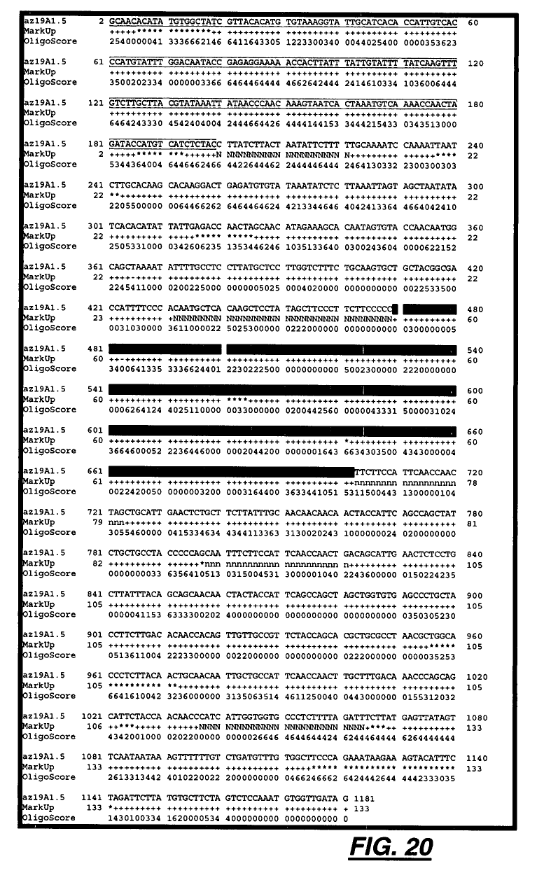

The following sequences were targeted for silencing: az19A1.2, az19A1.3, az19A1.4, az19A1.5, az19A1.6, az19A1.7, az19A2.1, az19A2.2A. FIG. 20 illustrates alignment of the best target sequence with a match-up key and an Oligo score. The key is coded as follows: “*” indicates that the sequence matches all sequences chosen for silencing; “+” indicates that the sequence matches at least one other sequence chosen, “−” indicates that the sequence does not match any other sequence, “N” indicates the sequence matches sequences that should NOT be silenced, and “n” indicates that the sequence matches sequences that should NOT be silenced and match is outside of corresponding aligned sequence. The Oligo score is computed for the oligo that starts at the given position. Scores range from 0 to 6.

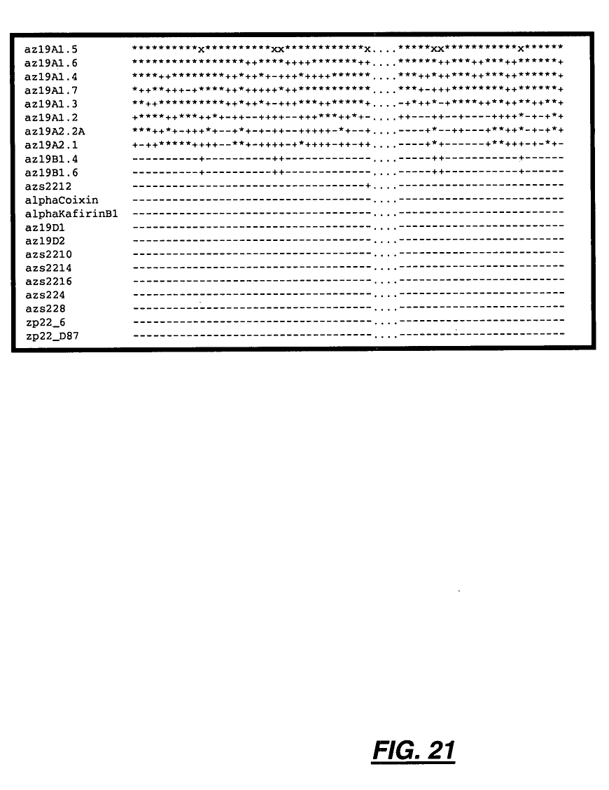

FIG. 21 shows a cartoon representation of an alignment. Each character represents 21 base pairs (b). A “*” indicates all match, a “+” indicates that one or more match (but not all match), a “−” indicates that none match, and a “.” indicates that there is a gap.

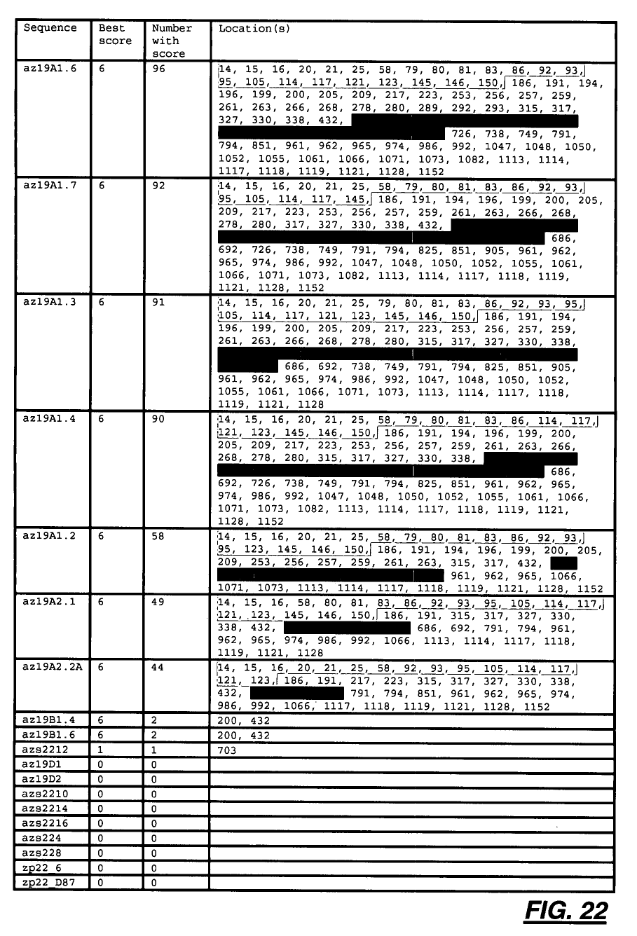

FIG. 22 is a table illustrating the best match. Note the locations are also selected.

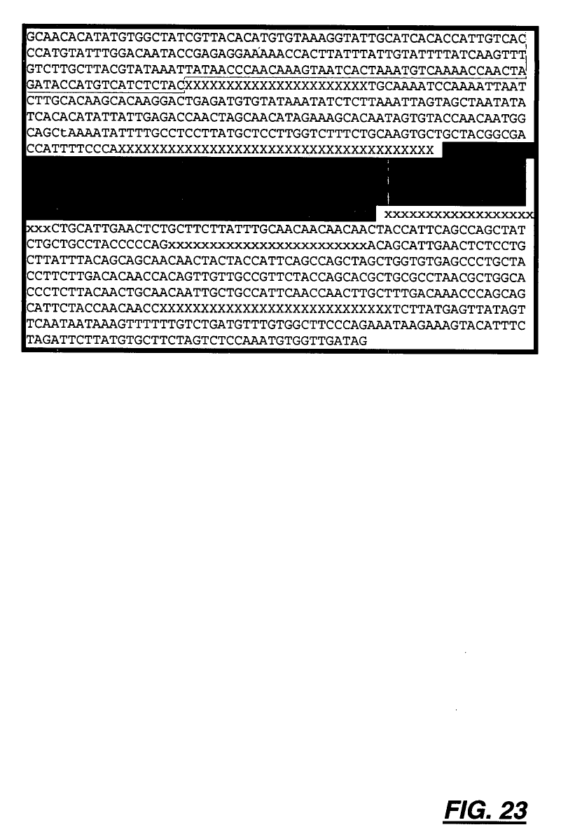

FIG. 23 illustrates az19A1.5 marked for matches. An uppercase letter indicates that there is a match. A lower case letter indicates that there is no match. An “X” or “x” indicates there is a negative match.

19 kDa-B Class

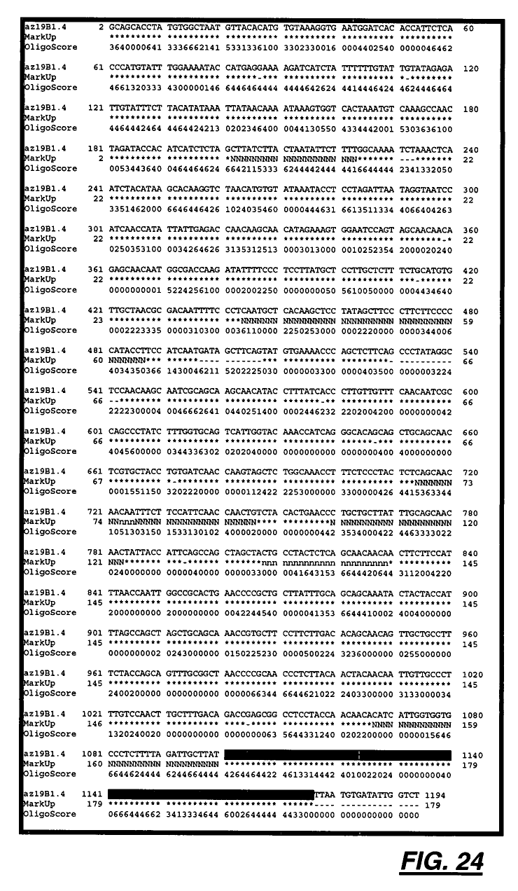

Sequences were also targeted for silencing, including az19B1.4 and az19B1.6. Alignment of the best target sequence with a match-up key and an Oligo score are shown in FIG. 24.

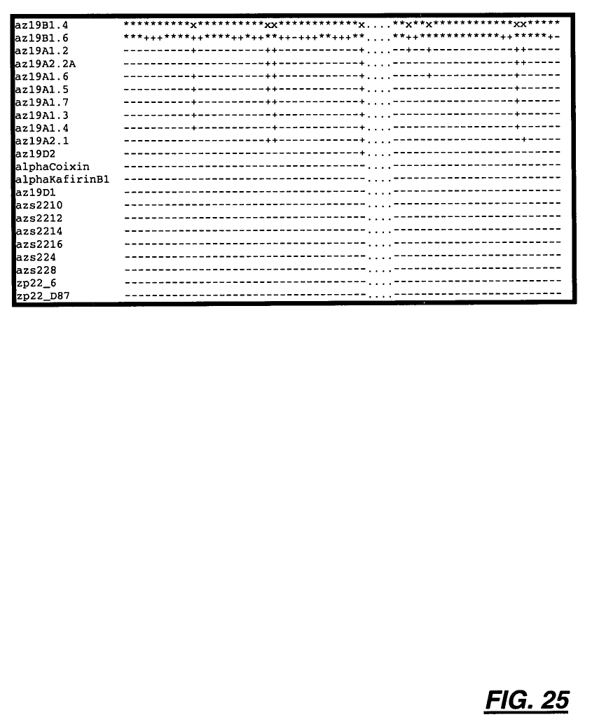

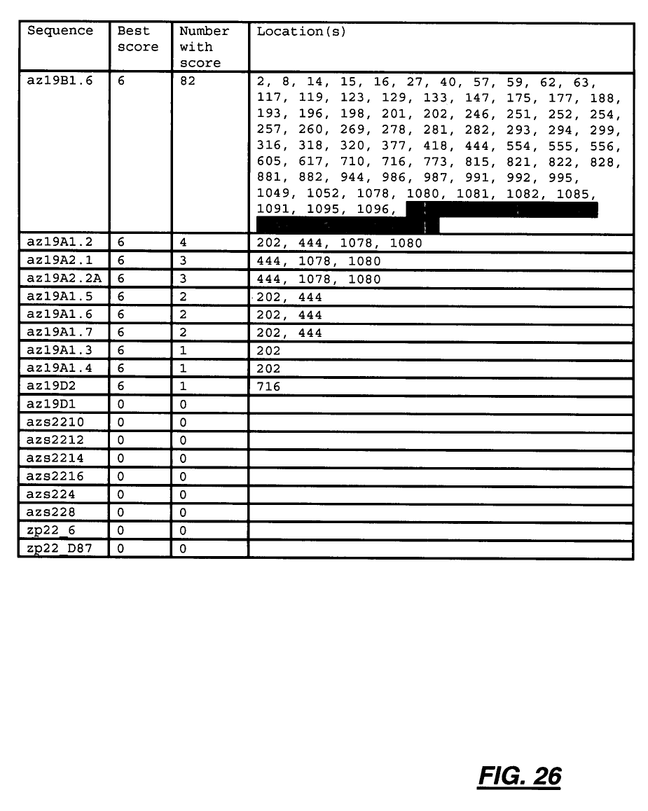

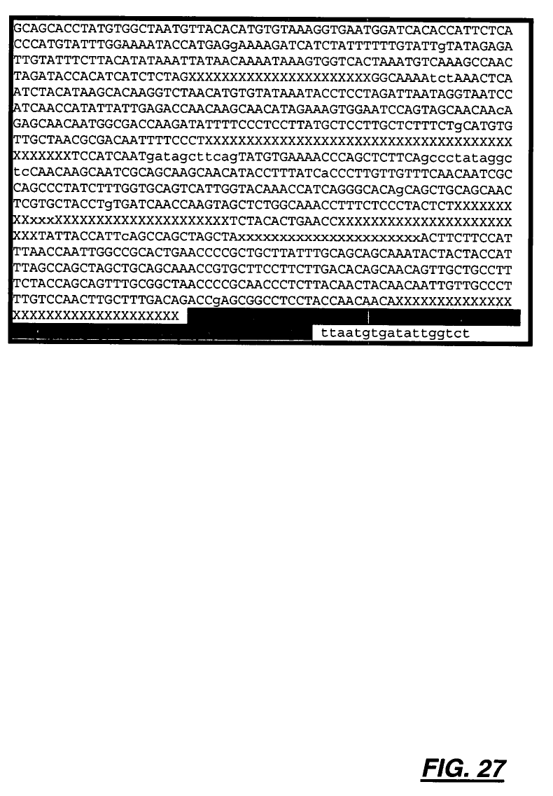

FIG. 25 provides a cartoon alignment. FIG. 26 is a table illustrating the best match. FIG. 27 illustrates az19B1.4 marked for matches.

19 kDa-D Class

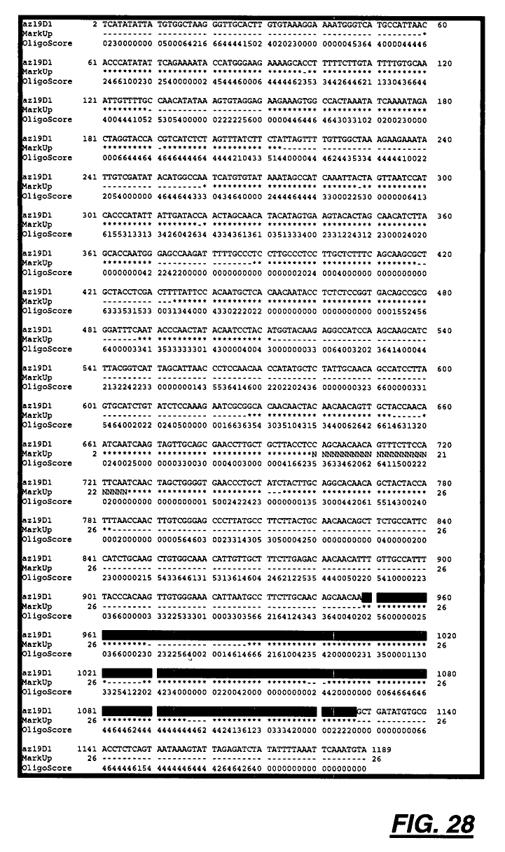

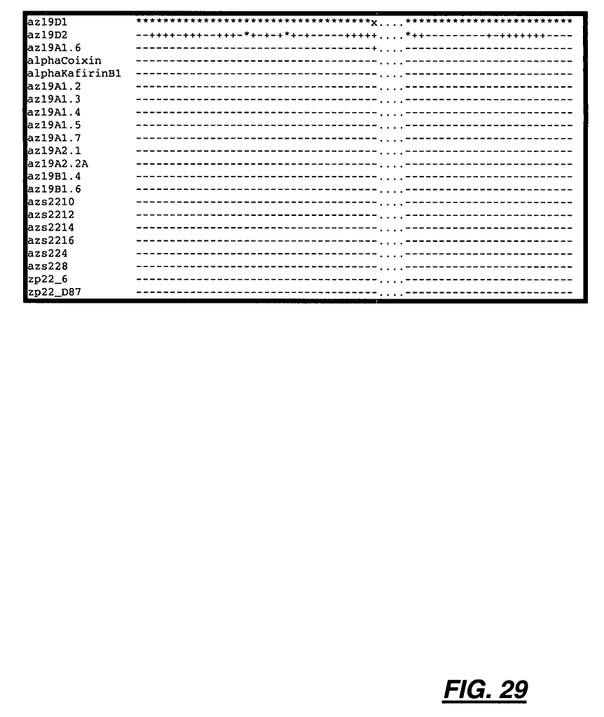

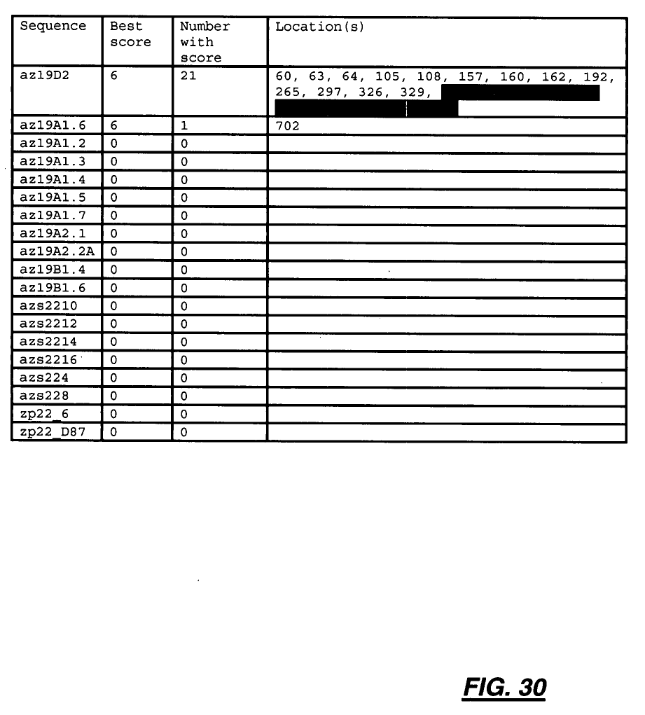

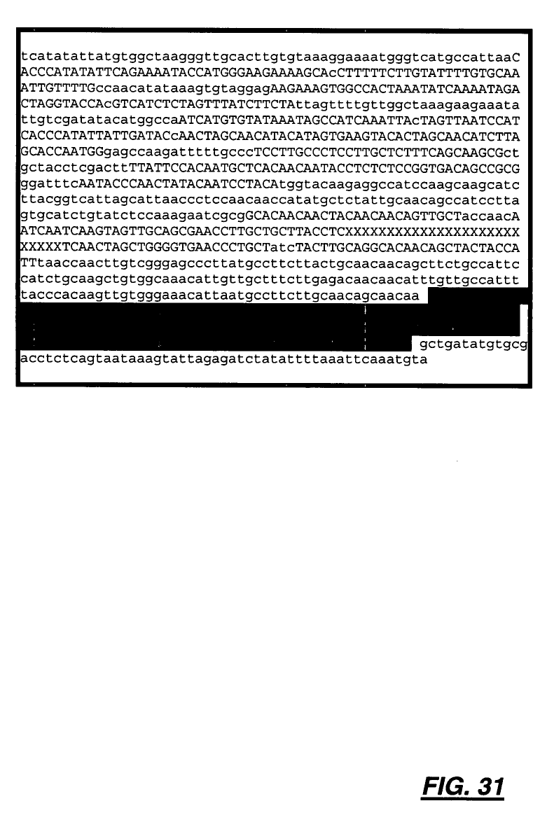

Next, az19D1 and az19D2 sequences were targeted for silencing. Alignment of the best target sequence with a match-up key and an Oligo score are shown in FIG. 28. FIG. 29 illustrates a cartoon alignment. FIG. 30 is a table illustrating the best matches. FIG. 31 illustrates az19D1 marked for matches.

22 kDa-FL2

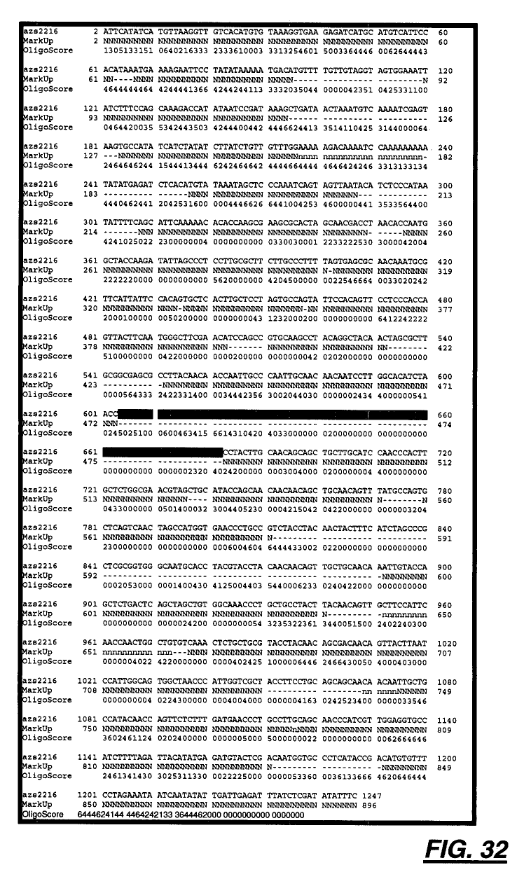

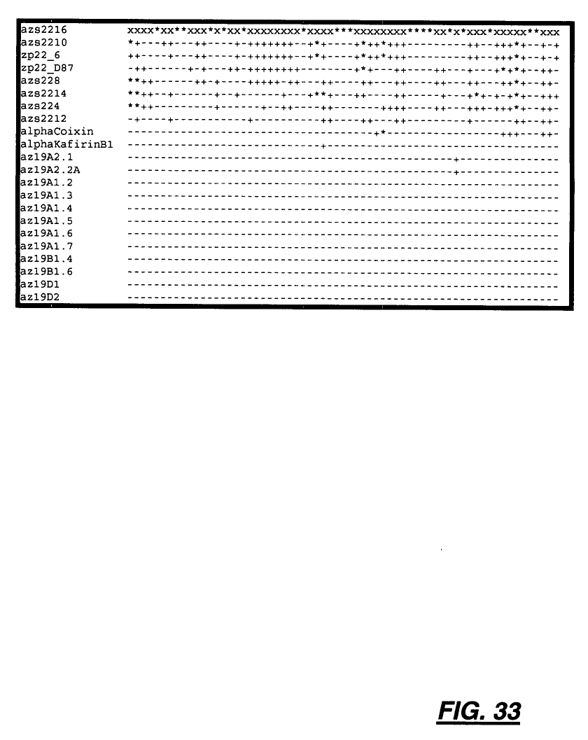

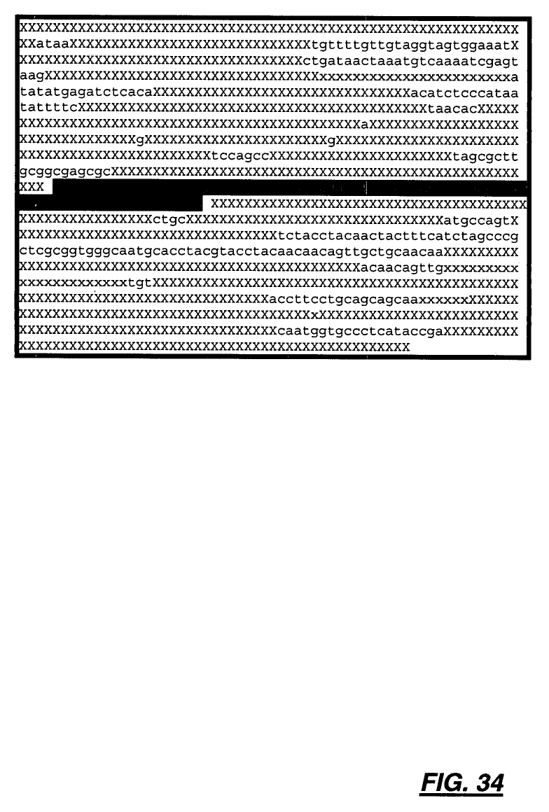

The azs2216 sequence was targeted for silencing. Alignment of the best target sequence with a match-up key and an Oligo score are shown in FIG. 32. FIG. 33 provides a cartoon alignment. FIG. 34 illustrates azs2216 marked for matches.

Thus, a method for identifying one or more polynucleotide sequence for specifically silencing a target gene has been provided. The method may be used to identify a sequence for use in silencing applications that specifically silences a target gene. The method can mimic a plant cell's in vivo silencing process. The method may reduce the possibility of silencing non-target genes, their mRNA, thereby minimizing off-target effects on non-targeted genes or their mRNA. Thus, the method can increase research efficiency by facilitating the selection of polynucleotide sequences for specifically silencing a target gene. This can be advantageous in that the method may allow one to conserve resources that would otherwise be diverted to selecting and utilizing sequences that are ineffective for specifically silencing a target gene. This can further be advantageous in that the method can provide an increase the repertoire of available sequences that can be used for the silencing applications, thereby creating a larger pool from which to choose the better or best subsequences for silencing. The method can further facilitate the selection of the most effective sequences for specifically silencing a target.

In addition, user interface and a method for providing a user interface that provides for synchronized selection of candidate sequence regions in a plurality of views to assist a user in understanding the data presented. The method can present information to the user in a manner more conducive to a user making correct decisions quickly and conveniently. It should be understood that the present invention is not to be limited to the specific disclosure provided herein. In fact, the present invention contemplates numerous variations in the particular method steps, the type of scoring, the size of window, the implementation of the method, the user interface where used, and other variations.

What is claimed is:

1. A method of identifying one or more polynucleotide sequence for specifically silencing a target gene comprising:
providing a target polynucleotide sequence to be silenced;
processing said polynucleotide sequence into a series of polynucleotide subsequences;
comparing each polynucleotide subsequences to said target sequence to obtain a percent identity for each subsequence;
comparing said percent identity of each subsequence to a threshold percent identity value;
selecting each polynucleotide subsequence that meets or exceeds the threshold percent identity value;
scoring each polynucleotide subsequence for potential silencing efficacy of the target polynucleotide to obtain a score; and
reporting the subsequences that meet or exceed the threshold percent identity value and the score for each polynucleotide sequence that meets or exceeds the threshold percent identity value to thereby assist in identifying one or more polynucleotide subsequences for specifically silencing a target gene.

2. The method of claim 1 further comprising providing a non-target polynucleotide sequence that is not to be silenced.

3. The method of claim 1 further comprising processing said polynucleotide sequence into a series of polynucleotide subsequences using a sliding window analysis to obtain subsequences of the same length.

4. The method of claim 1 further comprising preselecting a threshold percent identity value.

5. The method of claim 1 further comprising analyzing each polynucleotide subsequence for potential silencing efficacy of a target polynucleotide using an algorithm, wherein said algorithm has a parameter that takes into consideration one or more physical characteristics of the subsequence selected from the group consisting of: melting temperature (Tm), the nucleotide content of the 3′ overhangs, the length of the subsequence, the nucleotide distribution over the length of the subsequence, nucleotide end-composition of the target site and presence and location of mismatches with respect to a reference sequence, base composition at the 5′ end of the RNA molecule, helix stability, base composition numbers at the 3′ end, and the free energy of the molecule.

6. The method of claim 1 further comprising ranking the subsequences that meet or exceed the threshold percent identity value.

7. The method of claim 6 wherein the step of ranking being at least partially based on score.

8. The method of claim 1 further comprising ranking the identified subsequences that meet or exceed the threshold percent identity value in comparison to the target sequence and score according to the score and higher threshold percent identity value and subsequences that are below the threshold percent identity value in comparison to the non-target sequence.

9. The method of claim 1 wherein the step of scoring occurs prior to obtaining a percent shared identity.

10. The method of claim 1 wherein the step of scoring occurs after obtaining a percent shared identity.

11. The method of claim 1 further comprising adding nucleotides to an identified subsequence.

12. The method of claim 1 wherein said polynucleotide sequence is a cDNA sequence, a genomic DNA sequence, or an RNA sequence.

13. The method of claim 1 wherein said polynucleotide subsequence is a DNA sequence or an RNA sequence.

14. The method of claim 1 further comprising generating a nucleic acid molecule comprising the identified subsequence.

15. The method of claim 14 further comprising transforming a plant with a nucleic acid molecule comprising the identified subsequence.

16. A method of identifying one or more polynucleotide sequence for specifically silencing a target gene comprising:
providing a target polynucleotide sequence to be silenced;
determining a plurality of polynucleotide subsequences from the target polynucleotide sequence;
determining a percent identity between each of one or more of the plurality of polynucleotide subsequence and a reference sequence;
scoring each of the plurality of polynucleotide subsequences for potential silencing efficacy to provide a score for each of one or more of the plurality of polynucleotide subsequences;
reporting the score and the percent identity for at least one of the plurality of polynucleotide subsequences.

17. The method of claim 16 wherein the plurality of polynucleotides being determining by applying a sliding window to generate the plurality of polynucleotide subsequences.

18. The method of claim 16 wherein the reference sequence being determined from the target polynucleotide sequence.

19. The method of claim 16 wherein the reference sequence being determined from a library.

20. The method of claim 16 wherein the score is an overall score based on a plurality of separate scoring algorithms.

21. The method of claim 16 further comprising ranking at least a subset of the plurality of polynucleotide subsequences.

22. A computer-implemented method of identifying one or more polynucleotide sequence for specifically silencing a target gene comprising:
receiving a selection of a target polynucleotide sequence to be silenced from a user;
determining a plurality of polynucleotide subsequences from the target polynucleotide sequence;
determining a percent identity between each of one or more of the plurality of polynucleotide subsequence and a reference sequence;
scoring each of the plurality of polynucleotide subsequences for potential silencing efficacy to provide a score for each of one or more of the plurality of polynucleotide subsequences;
providing an output to the user indicating the score for each of the one or more of the plurality of polynucleotide subsequences.

23. The computer-implemented method of claim 22 further comprising receiving a selection of one of the plurality of polynucleotide subsequences from the user.

24. The computer-implemented method of claim 23 further comprising marking up the target polynucleotide sequence using the selection of the one of the plurality of polynucleotide subsequences from the user to provide a markup sequence.

25. The computer-implemented method of claim 24 further comprising displaying the markup sequence.

26. A method of providing a user interface, comprising:
providing a display having (a) a first region adapted for displaying an identifier for each of a plurality of sequences and a score for each of the plurality of sequences, and (b) a second region adapted for displaying a markup sequence formed by marking up a target polynucleotide sequence with one of the plurality of sequences;
receiving a selection of one of the plurality of sequences from a user;
updating the second region with the selection of the one of the plurality of sequences to display marking up of the target polynucleotide sequence with the selection of one of the plurality of sequences from the user.

27. The method of claim 26 wherein the display further includes a third region adapted for displaying a cartoon representation for each of the plurality of sequences.

28. The method of claim 27 wherein the display further includes a fourth region adapted for displaying an alignment for the selection of the one of the plurality of sequences.
